(19) United States
(12) Patent Application Publication
(10) Pub. No.: US 2007/0212693 A1
(43) Pub. Date: Sep. 13, 2007

(54) FACTOR RECA FROM BACILLUS LICHENIFORMIS AND RECA-INACTIVATED SAFETY STEMS USED FOR BIOTECHNOLOGICAL PRODUCTION

(75) Inventors: Jorg Feesche, Erkrath (DE); Friedhelm Meinhardt, Senden (DE); Hannes Nahrstedt, Braunschweig (DE); Jens Waldeck, Munster (DE); Mark Grone, Mainz (DE); Renee Charlott Eichstadt, Koln (DE)

Correspondence Address: CONNOLLY BOVE LODGE & HUTZ, LLP, P O BOX 2207, WILMINGTON, DE, 19899, US

(73) Assignee: HENKEL KGaA, DUSSELDORF (DE)

(21) Appl. No.: 10/593,425

(22) PCT Filed: Feb. 16, 2005
(86) PCT No.: PCT/EP05/01543

§ 371 Date: Dec. 27, 2006

(30) Foreign Application Priority Data
Mar. 19, 2004 (DE) 10 2004 013 988.1

(51) Int. Cl. C12Q 1/68; G01N 33/554; C07H 21/04; C12N 1/21; C12N 15/74; G01N 33/569; C07K 14/32

(52) U.S. Cl. 435/6; 435/7.32; 435/69.3; 435/252.31; 435/471; 530/350; 536/23.7

(57) ABSTRACT

The invention relates to the factor RecA from bacillus licheniformis DSM 13 (SEQ ID NO: 2), along with the associated gene recA (SEQ ID NO: 1), including related proteins and genes thereof, such as the variant indicated under SEQ ID NOS: 31 and 32, among others. According to the invention, gene recA is used for constructing gram-positive bacterial safety stems for biotechnological production, among other things, by inactivating the same in the respective stems. In a special embodiment, said stems are provided with additional functional deletions in phase-IV sporulation genes, preferably in gene SpoIV (in Bacillus licheniformis), gene yqfD (in B. subtilis), or the respective gene that is homologous thereto if said stems are naturally able to form spores. Furthermore, the inventive RecA represents a protein which can be used in molecular biological assays or for modulating the molecular biological activities of cells, especially in connection with DNA polymerization or recombination processes.

The present invention relates to the Factor RecA from Bacillus licheniformis DSM 13 as well as microorganisms as safety strains for biotechnological production, characterized in that they exhibit functional deletions in the associated gene recA. Furthermore, RecA is thereby available for further molecular biological preparations.

The present invention is in the field of biotechnology, in particular the manufacture of valuable substances by the fermentation of microorganisms that are capable of forming the valuable substances of interest. They include, for example, the manufacture of low molecular weight compounds, e.g. food supplements or pharmaceutically relevant compounds, or proteins, for which, as a result of their diversity, there again exists a large range of industrial applications. Firstly, the metabolic properties of the microorganisms in question are exploited and/or changed; secondly, cells are introduced that express the genes of the proteins of interest. Therefore, both cases mainly concern genetically modified organisms (GMO).

There exists a comprehensive prior art covering the fermentation of microorganisms, particularly also on the industrial scale; it ranges from the optimization of the strains in question with regard to the rates of formation and the nutrient utilization through the technical design of the fermenter to the recovery of valuable materials from the cells in question and/or the fermentation medium. Both genetic and microbiological as well as process engineering and biochemical approaches are involved. The object of the present invention is to improve this process in regard to the relevant safety aspects of the added microorganisms, namely on the level of the genetic properties of the strains under consideration.

The background is that the use of genetically modified organisms is generally subject to draconian legal guidelines with respect to biological safety. In most countries the operators of units with GMO are obliged to ensure that there is no possibility for the GMO to reach the environment. In addition, GMOs used for production should possess properties that—in the case that they ever were to reach the environment—are intended to make difficult or depending on the danger level even make impossible their reproduction (“concept of containment”).

As a result of the review article “Suicidal genetic elements and their use in biological containment of bacteria” by S. Molin et al. (Annu. Rev. Microbiol., 1993, volume 47, pages 139 to 166), both fundamental strategies are differentiated into “active” components, where controlled suicide systems are incorporated into the cells, or “passive” systems, where the cell properties are modified in such a way that their chance of survival under conditions of stress are reduced. The second relevant strategy for the present application is also described therein as the “disablement approach”.

GMO strains with a reduced risk for humans and the environment in the case of an unintentional release are designated as safety strains. Depending on the fundamental properties of the microorganisms, increasingly more properties are required that all represent a safety aspect. Consequently, it is advantageous to have available various instruments for the preparation of safety strains. Among these, some “passive” systems are already described in the prior art.

Thus, the application EP 369817 A1 relates to Bacillus strains, particularly B. subtilis, for the manufacture and secretion of proteins, in which the genes for extra cellular and intracellular proteases, namely rp-I, rp-II, isp-1, apr and/or npr have been functionally inactivated by point mutations or insertions of inactive gene copies. The sense of these genetically engineered modifications is to minimize protease activities that are harmful for the proteins of interest that are manufactured with these strains. The strains in question can additionally dispose of mutations that inhibit the sporulation and consequently the formation of similarly harmful sporulation proteases. Below, the active gene in the null phase of the sporulation (see below) of B. subtilis is called spoOA, the inactivation of which inhibiting the formation of intracellular proteases linked with the sporulation.

The application WO 92/16642 A1 pursues the same method of resolution: It discloses that by inactivating the protease genes apr, npr, isp-1, epr, bpr, rsp and mpr from Bacillus, a major part of the extra cellular protease activity is switched off, and teaches that this can be further improved by inactivating the newly described gene vpr for the residual protease III. The possibility of inactivating spoOA so as to inhibit the formation of intracellular proteases is also noted here.

The sporulation of gram-positive bacteria concerns a development process for the formation of resting forms—the so-called spores—for outlasting adverse environmental influences. It is controlled by a complex regulatory cascade with probably more than 100 genes and with the participation of specific sigma factors. The interrelationship of this process with the cell cycle of B. subtilis is described, for example in the publication “Cell cycle and sporulation in Bacillus subtilis” (1998) by P. A. Levin and A. D. Grossmann in Curr. Opin. Microbiol., volume 1, pages 630 to 635. Here, mainly the transcription factor SpoOA is presented as the control element for triggering the sporulation. The review article “Control of sigma factor activity during Bacillus subtilis sporulation” (1999) by L. Kroos et al. in Mol. Microbiol., volume 31, pages 1285 to 1294 summarizes the sequential activation of the phase specific genes by various sigma factors. In this process their sequences are observed after the successive stages null and then I to VII. This numerotation is also found again in the identifiers of the participating genes and factors.

The application EP 492274 A2 discloses that in the prior art the inactivation of sporulation genes was achieved already for non-specific mutagenesis, whereby asporogenous mutants (spo-minus phenotype) were obtained. EP 492274 A2 itself describes a spoIID treated B. subtilis strain from targeted mutagenesis in the early sporulation gene, which, with a reversion frequency of less than 10⁻⁸, is practically no longer capable of forming spores. This application teaches the use of this strain, first after inactivation of the additional genes leu (for the leucine synthesis), pyrD1 (for the uracil synthesis), apr and npr for the manufacture of valuable products for biotechnological production, because advantages in the production as well as safety aspects are linked therein.

The application WO 97/03185 A1 also relates to the inactivation of the sporulation capability of Bacillus species, with the exception of B. subtilis, and the use of these strains for the biotechnological manufacture of valuable products. According to this application, the early encoding gene spoIIAC for the sigma factor F should be functionally inactivated, advantageously in combination with deletions in genes of the likewise activated sporulation gene groups spo2, spo3. For this, an irreversible inactivation of the relevant chromosomal segments for spoIIAC is described.

The application WO 02/097064 A1 (EP 1391502 A1) relates to microorganisms, in which the genes from the stages II, III, IV or V of the sporulation have been deleted or inactivated. They concern the genes sigE, sigF, spoIIE, spoIISB and sigG of B. subtilis, which reside within the locus of spoIVCB to spoIIIC of B. Subtilis. Using the databank SubtiList (available on http://qenolist.pasteur.fr/SubtiList/genome.cqi), this can be narrowed down to the region of the positions from ca. 2 642 000 kb to ca. 2 700 000 kb of the total genome of B. subtilis, which has since become known. The object of this application was based on the elimination of superfluous or harmful activities of bacillus strains in order to improve the biotechnological production. By modifying the middle to late sporulation genes in this way, the use of the strains in question represses spore formation for the biotechnological production; this would have an advantageous effect on the nutrient utilization and energy utilization; the fermentation time could be simultaneously increased, thereby increasing the total yield of interesting valuable products.

The transition of gram-positive bacteria into the resting form of the spores can also be triggered by unfavorable environmental conditions. Exactly this should then occur when bacteria accidentally escape from the optimal growth conditions of fermentation in the equipment and reach the surroundings. In contrast, as has just been set forth, up to now the prevention of the sporulation capability for producing safer GMOs has only received little consideration. The relevant prior art only seems to suggest that the sporulation of gram-positive bacteria should be completely prevented at an early stage a) because of the associated protease activities and/or b) to prolong the fermentation period, in order to ultimately enhance the fermentation yield of the thus obtained asporogenous strains. In contrast, only some additionally introduced mutations are disclosed for pursuing safety aspects.

The encoding gene recA for the factor recA described in procaryotae is well known in molecular biology, up to now, however, in another context than for the manufacture of safety strains. This factor binds specifically and cooperatively to single stranded DNA and provides for a partial unwinding of double stranded DNA by ATP hydrolysis. This procedure enables the genetic recombination process, i.e. the exchange of strands between similar DNA molecules. Thus, in molecular biology, it is a standard procedure to use the recA gene in such a way that it inactivates by a suitable genetic construction with a defective recA copy and thereby a recA-minus-phenotype is produced that is no longer capable of recombination. According to U.S. Pat. No. 4,713,337, for example, deletion mutants produced by crossing over are genetically stabilized by subsequent inactivation of recA.

Thus, references to recA emerge in the most varied molecular biological contexts. For example, DE 1001 1358 A1, which deals with L-form bacterial strains, further mentions, besides numerous other possible modifications, the possibility inter alia of also mutating recA in order to achieve an improved transformation and plasmid stability.

A biochemical description of the RecA from Escherichia coli is provided, for example, in the publication “C-terminal deletions of the Escherichia coli RecA” by S. L. Lusetti et al. (2003; J. Biol. Chem., Volume 278, Book 18, pages 16372-16380). It emerges from this that the C-terminus of this molecule particularly interferes with the single strand binding and corresponding deletion mutants exhibit, besides other biochemical properties, an increased mitomycin sensitivity in this region. It is well known that mitomycin interferes with the DNA synthesis and thus acts as a bactericide. In contrast, the N-terminus is more strongly involved with the binding of DNA double strands.

The review article by S. Molin et al., cited above, also refers to work in which a recA-minus mutation is used as the marker gene for gram-negative Escherichia coli. It was surmised that this mutation alone could be sufficient to eliminate all environmental risks by this strain. On the other hand, two disadvantages of this approach are discussed, namely that it would be technically difficult to manufacture these mutants, and secondly the strain in question would also be hindered in its short-term competitive properties in such a way that one would prefer other limited viability mutations mentioned in the relevant article. Also, combined with the fundamentally different approach for producing safety strains, namely the introduction of suicide systems, the inactivation of Reca has turned out to be disadvantageous.

The publication “Freisetzung gentechnisch veränderter Bakterien”“Release of genetically modified bacteria”, by Selbitschka et al. (2003; Biologie in unserer Zeit, volume 33, book 3, pages 162-175) describes the release of gram-negative bacteria of the species Sinorhizobium meliloti, modified with a luciferase gene, in a multi-year outdoor test. They additionally carried an inactivation of the recA gene that led to the fact that cells of these clones could not ultimately survive under natural conditions.

In the course of his thesis entitled “Entwicklung eines Sicherheitsstammes von Bacillus megaterium DSM 319 und molekulargenetische Charakterisierung des Gens für die extrazelluläre neutrale Metalloprotease (nprM)”, (Development of a safety strain of Bacillus megaterium DSM 319 and molecular genetic characterization of the gene for the extra cellular neutral metalloprotease nprM), submitted to the Westfalian Wilhelms University Munster in 1995, K.-D. Wittchen produced a strain of gram-positive B. megaterium that comprises, after targeted gene disruption, deletions in the neutral metalloprotease (mentioned in the title), that of the isopropyl maleate-dehydrogenase, and a not more closely described Spo/V-protein. Finally, a recA mutation was conducted on this strain, which, however, produced no significant differences in regard to the UV-sensitivity of the strain in comparison with the wild type, but did during growth on mitomycin C-containing agar plates. This fourfold mutant was proposed as a safety strain, without however any investigation of its viability or even the practical consequences of this modification on a production process with suitably modified bacterial strains.

The Master's thesis of H. Nahrstedt (2000) from the same research group entitled “Molekulargenetische Charakterisierung des recA-Gens von Bacillus megaterium DSM 319 und Konstruktion einer Deletionsmutante”, “Molecular genetic characterization of the recA gene of Bacillus megaterium DSM 319 and construction of a deletion mutant” proposed the following, juxtaposing four mutations partly present in groups of genes: recA-minus, protease-minus, leucin-auxotrophie and sporulations-deficiency. It was discussed to introduce a recA deficiency as a safety marker in addition to others in production strains because this leads firstly to an inhibition of undesired recombination processes and also the mutants in question exhibited an increased sensitivity towards DNA-damaging agents i.e. they should have a lower chance of survival in the environment. This proposal was also not pursued further.

One can summarize the prior art for RecA to the effect that up to now this protein is predominantly known from genetic contexts. Up to now, the use of this factor and/or the inactivation of the gene in question for the production of safety strains of GMOs has been, if anything, rejected due to its physiological significance. Successfully described examples of this are merely recA-minus mutants of the gram-negative species Sinorhizobium meliloti and the gram-positive Bacillus megaterium, the latter only in combination with three further safety relevant mutations.

Overall, it can be considered that various alternative genetic systems based on a “passive” mode of action have been established for the manufacture of safety strains for the biotechnological production, such as the inactivation of protease genes, the exclusion of various metabolic genes for producing amino acid auxotropes or nucleobase auxotropes. For spore-forming gram-positive bacteria, the prevention of sporulation has been described, in particular at an early stage, principally, however, to obtain additional advantages in the fermentation. For this, it is considered advantageous to dispose of a plurality of differently acting systems in order to place them beside each other on a specific strain, thereby ensuring that the strain can be particularly reliably classified.

Accordingly, the object of the invention is to develop a further suitable safety system for genetically modified gram-positive bacteria, the basis for which being firstly the identification of a suitable factor and/or a suitable gene.

After having determined the fundamental suitability of such a system, one aspect of this object is represented by the isolation of a utilizable genetic element for it, possibly a gene, and the amino acid sequence of an optionally coded factor thereof, to provide this system suitable molecular biological constructions for use in production strains, particularly in combination with one or a plurality of additional regulation mechanisms that contribute to the safety.

A further aspect of the object is that this system should be combinable with other safety systems.

There was therefore a subtask of defining a further combinable safety system of this type, preferably one that would require no further mutations in addition to both of these systems. In other words: a maximum of two of these mutations should be sufficient to produce a gram-positive safety strain, to fulfill to a large extent requirements for the reduction of viability in the environment, i.e. should lead to a minimal reversion rate. A number of less than four juxtaposed active systems means an increasingly lower amount of work for the manufacture of these strains.

A secondary aspect of this object is to find a safety system of this type that is not so specific as to prevent it also being used in other molecular biological approaches.

This object is achieved by the factor RecA with an amino acid sequence that is identical to at least 96% to the amino acid sequence in SEQ ID No. 2 or by the encoded nucleic acid for a factor RecA, whose nucleotide sequence is identical to at least 85% of that of the nucleotide sequence given in SEQ ID No. 1.

The amino acid- and nucleotide sequences given in SEQ ID No. 2 and 1 are those for RecA. All positions from 1 to 1047 encode for the protein; the last three represent the stop codon. They are designated as the gene and protein recA respectively RecA. They originate from the strain Bacillus licheniformis, deposited under the number DSM 13 at the Deutschen Sammlung von Mikroorganismen und Zellkulturen GmbH, (German Collection of Microorganisms and Cell Cultures) Mascheroder Weg 1b, 38124 Braunschweig (http://www.dsmz.de). Inventive solutions to the problem are represented by all factors or nucleic acids that exhibit a sufficient homology to the defined percentages.

The corresponding factor from B. amyloliquefaciens can be regarded as the closest prior art. The associated DNA sequences and amino acid sequences have been published in the NCBI databank of the National Institute of Health, USA (http://www.ncbi.nlm.nih.gov) under the entry number AJ515542. RecA from B. licheniformis DSM 13 exhibits a homology on the amino acid level of 94% identity and on the nucleic acid level an identity agreement of 81.2%. Both comparisons emerge from the alignments of FIGS. 1 and 2, where the sequences of B. amyloliquefaciens are each presented in the second line.

RecA from B. subtilis and RecE from B. subtilis were determined as the closest similar enzymes, each with 93.4% identity. On the DNA level they exhibit homology values of 81.0% and 81.2% identity respectively. They are also published in the NCBI databank under the respective entry numbers Z99112 (region 161035 to 162078) and X52132. The comparisons of amino acids and DNA with these factors are also illustrated in FIGS. 1 and 2 (lines 3 and 4 respectively).

Example 1 of the present application illustrates how additional factors RecA can be conveniently obtained that conform to the identified homology region. A molecular biological procedure is shown there, according to which, relevant genes or gene segments can be obtained from chromosomal DNA preparations of the relevant species by means of specific PCR primers, in particular the oligonucleotides disclosed therein (SEQ ID NO. 25 to 30). If these primers cannot be successfully employed, then optionally similar primers can be employed in which individual positions can be varied as a function of the reaction conditions in the primer synthesis. In the case that only partial sequences have been obtained by the use of suitable primers (see FIG. 3B) then these PCR products can be assembled to contiguous DNA sequences using standard methods (exploiting overlapping). The amino acid sequence results directly from the encoded factor Reca obtained from the gene recA. Alternatively, the sequences disclosed in SEQ ID NO. 1 or SEQ ID NO. 31 can also be used as probes in order to isolate relevant genes from gene banks by the use of known methods.

From these high, required homology values for the factors described here, it can be expected that the same factor RecA assume the appropriate function, particularly in related strains or species, probably also in less related species, probably even in gram-negative organisms. According to the invention, this is in the DNA single strand binding discussed above and the associated role for recombination processes of nucleic acids. Comparable effects should also be linked with deletions of the recA gene, namely the prevention of DNA recombinations and thereby a reduced viability. At the same time, a recA gene from a strain should be suitable to take over this function in another; this is increasingly more successful with increasing similarity. In this manner the use of the gene in question is made possible for the manufacture of deletion mutants of the most varied gram-positive microorganisms.

Thus, a broad technological and commercially relevant field is opened up for RecA and principally for the associated gene recA, namely the manufacture of valuable products by the fermentation of genetically modified gram-positive bacteria. Their genetic stability and also their safety can be improved through mutations in recA. As shown further below, this is particularly true for strains that are actually employed in biotechnological production, such as, for example Bacillus licheniformis.

As described in more detail below, this occurs preferably in relation to and particularly preferably without further safety-relevant deletions. Likewise, this preferably occurs in the nearest possible related strains. However, this does not apply to B. megaterium, firstly because as previously described, the species' own recA genes or the deleted mutants present are available and secondly because this species that is characterized by its large cells and the resulting associated microbiological properties is usually not utilized for fermentation on an industrial scale.

Accordingly, the subjects of the present invention concern the factor RecA (SEQ ID No. 2) and the associated gene recA (SEQ ID No. 1) from B. licheniformis DSM 13 or its close relatives. The use of a recA gene for its functional inactivation in a gram-positive bacterium likewise represents an inventive subject matter, preferably in combination with the functional inactivation of an active gene in the phase IV of the sporulation of gram-positive microorganisms, preferably spoIV, yqfD or its homologs. Advantageously, this occurs with the help of genes spoIV and yqfd that are further described in the present application. The gram-positive microorganisms obtained in this way represent a corresponding subject of the present invention; likewise the fermentations carried out with these organisms, particularly for manufacturing valuable products. Furthermore, a RecA protein is made available by the present application and can be used in molecular biological approaches or for modulating the molecular biological activities of cells, particularly in relation to DNA polymerization procedures or recombination procedures.

The first inventive subject matter includes each factor RecA defined above containing an amino acid sequence that is increasingly preferably identical to at least 96.5%, 97%, 97.5%, 98%, 98.5%, 99%, 99.5% of the amino acid sequence listed in SEQ ID No. 2 and quite particularly preferably 100% identical.

As discussed, an increasing similarity is expected to provide an increasing functional match and thereby an interchangeability of the factors.

This preferably concerns a factor RecA that is coded by a nucleic acid whose nucleotide sequence is at least 85% identical to the nucleotide sequence listed in SEQ ID No. 1.

In preferred embodiments the factors are coded from a nucleic acid whose nucleotide sequence is increasingly preferably identical to at least 87.5%, 90%, 92.5%, 95%, 96, 97%, 98% of the nucleotide sequence listed in SEQ ID No. 1 and quite particularly preferably 100% identical.

The factors in question or genes for the transformation into other, preferably related species or for modifications are made available by the nucleic acids. In particular, they include, as discussed below in more detail, mutations of the genes in question. With an increasing degree of identity with the listed sequence, the success for such species should be all the more greater with increasing relation to B. licheniformis, particularly for the species B. licheniformis itself, which is particularly important for biotechnological production. Such nucleic acids are obtained as discussed above in example 1; also, reference has already been made to the isolation from gene banks.

As mentioned, principally the nucleic acids that encode a factor RecA and whose nucleotide sequence is at least 85% identical to the nucleotide sequence listed in SEQ ID No. 1, serve to realize the present invention.

This is all the more true for the kind of nucleic acids whose nucleotide sequence is increasingly preferably identical to at least 87.5%, 90%, 92.5%, 95%, 96%, 97%, 98%, 99% of the nucleotide sequence listed in SEQ ID No. 1 and quite particularly preferably 100% identical. They can be used in suitable constructions for the transformation and/or for mutagenesis, wherein an increasing similarity affords the greater probability of the desired success.

This quite particularly preferably concerns a nucleic acid of the type that encodes a previously described factor RecA. This is true for strategies in which a functional factor RecA should be manufactured, for example for the molecular biological approaches listed below, or for attaining a maximum match to the endogenous gene that actually encodes for Reca, which should be modified and/or switched off. In many cases a mutation suffices in a single position to switch off the gene or the factor in its natural function, for example through a nonsense mutation.

The use of nucleic acid that encodes for a factor RecA for the functional inactivation of the gene recA in a gram-positive bacterium that is not Bacillus megaterium represents an inventive subject matter.

Firstly, for B. megaterium there exist studies already mentioned in the introduction, in which it was proposed to delete recA at the same time as several other mutations so as to afford safety strains. Secondly, other gram-positive bacteria, such as for example those of the genera Bacillus, Staphylococcus, Corynebacterium and Clostridium, illustrate in the prior art the more important host organisms for the biotechnological production of valuable products (see below).

In the sense of the present application, functional inactivation is understood to mean all types of modification or mutation, whereby the function of a RecA as the single strand binding factor is prevented. This includes the embodiment where a practically complete, but inactive protein is formed, where inactive parts of Reca are present in the cell, up to the possibilities where the gene recA is no longer translated or even completely deleted. Thus the initially discussed “use” of this factor or this gene of this embodiment consists in the fact that the factor or gene from the relevant cells no longer functions naturally. In accordance with this inventive subject matter, this is achieved genetically, in that the gene in question is switched off.

According to an embodiment of this use, a nucleic acid that encodes for a non-active protein is introduced with a point mutation.

Nucleic acids of this type can be produced by the use of known point mutagenetic processes. Some are illustrated, for example in pertinent handbooks such as that from Fritsch, Sambrook und Maniatis “Molecular cloning: a laboratory manual”, Cold Spring Harbour Laboratory Press, New York, 1989. In addition, there are now commercial kits available for this, for instance the QuickChange® kit of the Stratagene company, La Jolla, USA. The principal resides therein that oligonucleotides with single substitutions (Mismatch-Primer) are synthesized and hybridized with the provided single stranded gene; subsequent DNA polymerization then affords the corresponding point mutants. The respective species' own recA sequences can be used for this. Because of the high homologies it is possible and according to the invention particularly advantageous to carry out this reaction using the sequence listed with SEQ ID No. 1 or for example the other sequences in FIG. 2 from related species. These sequences may also serve to create corresponding Mismatch-Primers for related species, in particular using the identifiable conserved ranges in the alignment of FIG. 2.

According to an embodiment of this use, a nucleic acid with a deletion mutation or insertion mutation is incorporated, preferably comprising each of the boundary sequences, which comprise at least 70 to 150 nucleic acid positions, of the area encoding for the protein.

These processes per se are also familiar to the person skilled in the art. In this way it is possible to prevent the formation of a factor RecA by the host cell in that a part of the gene from a corresponding transformation vector is cut out via restriction endonucleases and the vector is then transformed into the host of interest, where, via the—up to then still possible—homologous recombination, the active gene is exchanged for the inactive copy. In the embodiment of the insertion mutation, the intact gene can simply be introduced to interrupt or instead of a recA gene part of another gene, for example a selection marker. The occurrence of the mutation can be phenotypically tested by known methods.

Such an approach was chosen in example 2: As mentioned therein, two flanking regions of each ca. 340 bp from SEQ ID NO. 31 were exploited in order to delete the intermediate part of the gene recA of a B. licheniformis strain (see FIG. 3B). In the following example 3, the success of this deletion is verified at the genetic level. FIG. 4 (A and B) proves that the DNA fragment under consideration has been suitably shortened by the deletion. The phenotypical description of the mutants obtained in this manner is presented in the following examples. According to this, recA-inactivated strains are significantly more UV sensitive than those with an intact recA gene (example 6).

In order to enable each of the required recombination occurrences between the defective gene introduced into the cells and the endogenously present intact gene copy, for example, on the chromosome, then according to the actual state of knowledge a match is required in respectively at least 70 to 150 relevant nucleic acid positions, respectively in both boundary sequences to the non-matching part, whereby it does not depend on the part lying between. Accordingly, those embodiments are preferred that simply comprise two flanking regions of at least these sizes.

According to an alternative embodiment of this use, nucleic acids with a total of two nucleic acid segments are incorporated that each preferably comprise at least 70 to 150 nucleic acid positions and thereby at least partially, preferably completely flank the area encoding for the protein.

Protein encoding segments are not inevitably needed to solely enable the exchange of both gene copies by homologous recombination. In fact, the boundary areas of the genes in question that naturally exert another function (promoter, terminator, enhancer etc.) or that represent merely non-functional intergenic segments are also suitable for this. Thus, the functional inactivation can also consist, for example, in the deletion of the promoter, whereby for a deletion mutation of this embodiment, one has to resort to flanking, non-encoding segments. In some cases it may also be judicious to select those segments for the flanking region, which partially reach into the protein encoding area and are partially located outside.

These regions that are at least partially non-encoding can be taken for example from SEQ ID NO. 31 for B. licheniformis. Those for the gene recA from B. amyloliquefaciens and for recA and recE from B. subtilis can be taken from the abovementioned data bank entries, for example. For other strains, for example also for recA from B. licheniformis, it is possible to access the relevant non-encoding areas from a preparation of genomic DNA using PCR-based methods; as is exemplified in example 1. These methods (for example anchored PCR with primers outwardly facing into an unknown region) are established in the prior art. The known gene segments serve as the starting points and serve to open up the still unknown regions. As soon as they have been sequenced after amplification they can themselves serve to synthesize further primers, and so on. According to the present invention, the required primers based on SEQ ID NO. 1 and 31 can also be created for other species of gram-positive bacteria, including in particular for those of the genus Bacillus, optionally by the introduction of variable positions as has been already been mentioned previously.

Consequently, a suitable use concerns one of the previously mentioned inventive nucleic acids and/or a nucleic acid whose nucleotide sequence matches with the nucleotide sequence listed in SEQ ID NO. 31 in the positions 369 to 1415 to at least 1045, preferably at least 1046, quite particularly preferably 1047 of these 1047 positions, or concerns the at least partially non-encoding flanking regions to these nucleic acids.

Thus, a comparison of both the nucleic acid sequences for example of SEQ ID NO. 1 and 31 shows that they differ in the region encoding for the protein in three positions (positions 369 to 1415 according to SEQ ID NO. 31). They are the positions 282, 283 and 284 for SEQ ID NO. 1 (CAC) and 650, 651 and 652 for SEQ ID NO. 31 (ACA). Both sequences fall under the above defined inventive homology region and characterize preferred embodiments of the aspect of the invention illustrated here: SEQ ID NO. 1 is based on the commercially available strain DSM 13; SEQ ID NO. 31 was obtained by improving the invention using principally any B. licheniformis strain (example 1). As they match in 1044 positions to SEQ ID NO. 31, the embodiment described in the examples as successful can be attributed an increasing preference for 1045, 1046 and quite particularly 1047 matching positions.

The various embodiments of this type are correspondingly preferred.

For the gram-positive bacterium, preferred embodiments of this use preferably concern the genera Clostridium or Bacillus and one that is naturally capable of sporulation, in which at the same time a gene from the phase IV of the sporulation is functionally inactivated with recA.

Many gram-positive bacteria, as described in the introduction, are capable of inducing the sporulation process under suitably unfavorable environmental conditions. According to the invention, this can be exploited as far as safety aspects are concerned, as a sporulation gene from the comparatively late phase IV of the sporulation is also functionally inactivated in combination with the above-illustrated inactivation of recA. In this way two combinable systems are simultaneously and inventively available for manufacturing safe GMOs corresponding to the formulated object. Up to the present, the combination of both systems was not yet known, particularly for this purpose.

The inactivation of sporulation genes in species of the genera Clostridium and Bacillus was particularly successful and therefore the characterizing embodiments of this are correspondingly preferred. The experiments for spoIV from B. licheniformis, illustrated in the examples of the present invention, conceptually follow the same molecular biological method as is described above for recA. Thus, according to example 1, the primers shown in SEQ ID NO. 19 to 24 were successfully used to obtain a spoIV gene from a B. licheniformis strain (see FIG. 3A). Example 2 shows the method for functional inactivation and example 3 its success (FIG. 4). Example 4 and FIG. 5 prove the expected phenotypical sporulation defects.

It is particularly surprising that the prevention of sporulation in such a late phase is so successful for this purpose. Indeed, spores (so-called “gray-phase spores”) are still formed under suitable conditions in spoIV mutants of B. licheniformis, however they are sterile and are no longer capable of germinating. In this regard this mutation accommodates the safety aspect. Up to now, a prevention of sporulation was rather favorized at an earlier point in time. The inactivation in phase IV additionally makes sure that the active factors in the earlier sporulation phases will also continue to be formed in the mutants. Without wishing to be bound by this theory, one can suppose that at least some of these cell factors are also required for the normal metabolic processes that take place during the fermentation. With their elimination at an earlier point in time, they were no longer available. Conversely, the advantageous effect from the inactivation of the sporulation in phase IV consists in that the resulting interference in the physiology of the cells is not so serious and the fermentation itself is less affected than for an earlier elimination of these genes. The growth curve determined in example 5 and illustrated in FIG. 6 demonstrates the success of this concept.

This type of use is preferred in which the inactivated gene from the phase IV sporulation in the nomenclature of B. subtilis is one of the genes spoIVA, spoIVB, spoIVCA, spoIVCB, spoIVFA, spoIVFB or yqfD or is a homologous gene to this, preferably in the case of B. subtilis is the gene yqfD, in the case of Bacillus licheniformis is the gene spoIV and all other cases is a homologous gene to this.

All of these genes are known per se and have been described for this sporulation phase. The B. subtilis gene spoIVA encodes for the phase-IV sporulation protein A that has been deposited in the databanks of Swiss-Prot (Geneva Bioinformatics (GeneBio) S. A., Geneva, Switzerland; http://www.genebio.com/sprot.html) and NCBI (see above) under the number P35149. It plays a role in the formation of an intact spore hull and its assembly. The amino acid sequence of the associated factor SpoIVA is listed in SEQ ID NO. 8 of the present application, in fact as the translation of the previous DNA sequence produced by the Patentln program. The associated nucleotide sequence can be found in the databank Subtilist of the Institute Pasteur, Paris, France (http://genolist.pasteur.fr/SubtiList/genome.cgi) under the number BG10275 and is listed in the sequence protocol in SEQ ID NO. 7, in fact with the 200 nucleotides situated before the 5′-end and the 197 nucleotides situated behind the 3′-end. Here, irrespective of the fact that these boundary sequences are likely to comprise completely meaningful genetic information, in particular regulation elements or also segments of other genes, the complete nucleotide sequence from 1 to 1876 listed under SEQ ID NO. 7 is described according to the invention as the gene spoIVA. The encoded region extends from the positions 201 to 1679; the first codon, i.e. the positions 201 to 203 are not translated in vivo as leucine but rather as methionine.

The B. subtilis gene spoIVB encodes for the phase IV sporulation protein B that is deposited in the databanks of Swiss-Prot and NCBI under the number P17896. It is relevant to the sigma factor K-dependent transition point during the sporulation or its activation in the mother cell. It plays a role in the intercompartmental signal transfer, probably over the hydrophobic N-terminal. The amino acid sequence of the factor SpoIVB is listed in SEQ ID NO. 10 of the present application, in fact as the translation of the previous DNA sequence produced by the Patentln program. The associated nucleotide sequence can be found in the databank Subtilist under the number BG10311 and is listed in the sequence protocol in SEQ ID NO. 9, in fact with the 200 nucleotides situated before the 5′-end and the 197 nucleotides situated behind the 3′-end. Here, irrespective of the fact that these boundary sequences are likely to comprise completely meaningful genetic information, in particular regulation elements or also segments of other genes, the complete nucleotide sequence from 1 to 1675 listed under SEQ ID NO. 9 is described according to the invention as the gene spoIVB. The encoding region extends from positions 201 to 1478.

The B. subtilis gene spoIVCA encodes for a putative site-specific DNA recombinase that is deposited in the databanks of Swiss-Prot and NCBI under the number P17867. It probably plays a role in recombining the genes spoIIIC and spoIVCB from which emerges the sigma factor K. The amino acid sequence of this recombinase is listed in SEQ ID NO. 12 of the present application, in fact as the translation of the previous DNA sequence produced by the Patentln program. The associated nucleotide sequence can be found in the databank Subtilist under the number BG10458 and is listed in the sequence protocol in SEQ ID NO. 11, in fact, together with the 200 nucleotides situated before the 5′-end and the 197 nucleotides situated behind the 3′-end. Here, irrespective of the fact that these boundary sequences are likely to comprise completely meaningful genetic information, in particular regulation elements or also segments of other genes, the complete nucleotide sequence from 1 to 1900 listed under SEQ ID NO. 11 is described according to the invention as the gene spoIVCA. The encoded region extends from the positions 201 to 1703; the first codon, i.e. the positions 201 to 203 are not translated in vivo as valine but rather as methionine.

The B. subtilis gene spoIVCB encodes for the RNA polymerase sigma factor K precursor that is deposited in the databanks of Swiss-Prot and NCBI under the number P12254. The remainder of this factor is encoded from the gene spoIIIC, which is ca. 10 kb away on the chromosome, the area in between being designated as the SKIN. Excision of this fragment in the immediately preceding sporulation phase yields the active sigma factor K that acts as the transcription factor. The amino acid sequence of the partial factor SpoIVCB is listed in SEQ ID NO. 14 of the present application, in fact as the translation of the previous DNA sequence produced by the Patentln program. The associated nucleotide sequence can be found in the databank Subtilist under the number BG10459 and is listed in the sequence protocol in SEQ ID NO. 13, in fact, together with the 200 nucleotides situated before the 5′-end and the 197 nucleotides situated behind the 3′-end. Here, irrespective of the fact that these boundary sequences are likely to comprise completely meaningful genetic information, in particular regulation elements or also segments of other genes, the complete nucleotide sequence from 1 to 868 listed under SEQ ID NO. 13 is described according to the invention as the gene spoIVCB. The encoding region extends from positions 201 to 671.

The B. subtilis gene spoIVFA encodes for the phase IV sporulation protein FA. This factor which is probably capable of forming a heterodimer with SpoIVFB (see below) likely fulfils the task of stabilizing these factors but thereby also simultaneously inhibiting them. Therefore, SpoIVFA is also already formed at an earlier time, probably in phase II. The amino acid sequence of SpoIVFA is deposited in the databanks of Swiss-Prot and NCBI under the number P26936. It is listed in SEQ ID NO. 16 of the present application, in fact as the translation of the previous DNA sequence produced by the Patentln program. The associated nucleotide sequence can be found in the databank Subtilist under the number BG10331 and is listed in the sequence protocol in SEQ ID NO. 15, in fact, together with the 200 nucleotides situated before the 5′-end and the 197 nucleotides situated behind the 3′-end. Here, irrespective of the fact that these boundary sequences are likely to comprise completely meaningful genetic information, in particular regulation elements or also segments of other genes, the complete nucleotide sequence from 1 to 1192 listed under SEQ ID NO. 15 is described according to the invention as the gene spoIVFA. The encoding region extends from positions 201 to 995.

The B. subtilis gene spoIVFB encodes for the phase IV sporulation protein FB. This is a membrane-associated metalloprotease that is probably responsible for processing prosigma K to sigma K; it is also already formed in phase II of the sporulation. The amino acid sequence of SpoIVFB is deposited in the databanks of Swiss-Prot and NCBI under the number P26937. It is listed in SEQ ID NO. 18 of the present application, in fact as the translation of the previous DNA sequence produced by the Patentln program. The associated nucleotide sequence can be found in the databank Subtilist under the number BG10332 and is listed in the sequence protocol in SEQ ID NO. 17, in fact, together with the 200 nucleotides situated before the 5′-end and the 197 nucleotides situated behind the 3′-end. Here, irrespective of the fact that these boundary sequences are likely to comprise completely meaningful genetic information, in particular regulation elements or also segments of other genes, the complete nucleotide sequence from 1 to 1264 listed under SEQ ID NO. 17 is described according to the invention as the gene spoIVFB. The encoded region extends from the positions 201 to 1067; the first codon, i.e. the positions 201 to 203 are not translated in vivo as leucine but rather as methionine.

Both of the preferred genes also emerge from the prior art. The DNA sequence and amino acid sequence of spoIV from B. licheniformis are deposited in the databanks of Swiss-Prot and NCBI under the number AJ616332. This factor was described by M. Grone as an essential factor for the sporulation of B. licheniformis in his Master's Thesis (2002) entitled “Arbeiten zur Herstellung einer sporulationsnegativen Mutante von Bacillus licheniformis” (Contributions to the manufacture of a sporulation negative mutant from Bacillus licheniformis) in the Department of Biology of the Westfalian Wilhelms-University Münster, Germany. The associated sequences also comprising part of the regulatory regions are listed in the present application under SEQ ID NO. 3 and 4. For these sequences, it should be noted that according to the invention, the region of the nucleotides 1 to 1792 is designated as the gene spoIV, whereby the actual SpoIV encoding segment comprises the positions 140 to 1336; the boundary sequences may again comprise other genetic elements like regulation elements or parts of other genes. Here, the first codon GTG of positions 140 to 142 is not translated in vivo as valine but rather as methionine.

In this work, reference is also made to the factor or the gene yqfD from B. subtilis, which, with a homology of 68% identity on the amino acid level, is considered to be the closest similar protein known to date. This factor is listed in the databank of Swiss-Prot under the number P54469; both the amino acid sequence as well as the DNA sequence, each with ca. 200 bp flanking regions are in the databank Subtilist under the number BG11654. The entry there states that it is indeed an unknown protein, but based on the existing sequence homologies, it could be considered as similar to the phase IV sporulation protein. The associated sequences can be found in SEQ ID NO. 5 and 6 of the present application. Concerning these sequences, it should also be noted that irrespective of the likewise listed and possibly other genetic elements comprised in the boundary sequences, according to the invention the region of the nucleotides 1 to 1594 is designated as the gene yqfD, the actual protein encoding segment comprising the positions 201 to 1397. Here, the first codon GTG of positions 201 to 203, as in spoIV from B. licheniformis, is not translated in vivo as valine but rather as methionine.

It can be expected that all other gram-positive microorganisms naturally capable of sporulation possess homologs with similar functions to the seven cited genes and factors derived there from. They should be immediately identifiable using known techniques through hybridization with the nucleic acids listed in the sequence protocol or, as already discussed above, through PCR-based approaches for the sequencing of the associated chromosomal segments of these microorganisms, in particular by means of both homologous sequences SEQ ID NO. 3 or 5, whereby a certain variance over the species borders is made possible.

One of these genes, preferably yqfD/spoIV or its homologs, is inactivated with recA in the production strain at the same time according to the invention in order to obtain there from the corresponding safety strains. Advantageously, corresponding to the above embodiments for deletion mutagenesis, the nucleic acids listed in SEQ ID NO. 3, 5, 7, 9, 11, 13, 15 or 17 are each used for the inactivation. In this way, it is not necessary to identify the homologous genes in question from each of the species used for the production. It can be expected here that these deletions will be the more successful the closer the species in question are related to B. subtilis or B. licheniformis. This should be linked to an increasing homology of the genes in question. For this reason the boundary sequences comprising ca. 200 bp are also listed in the sequence protocol, as in this way constructs can be formed, corresponding to the embodiments for recA, which comprise the regions comprising the at least 70 to 150 positions needed for a crossing-over in completely flanking regions and in this regard can also be employed with a certain probability of success for the deletion of the segments under consideration in microorganisms that have not been completely characterized.

According to the invention, it is possible to inactivate, together with recA, several of the mentioned phase IV genes, thereby obtaining safety strains that besides being incapable of RecA-effected DNA recombination are incapable of forming mature spores. According to the invention, it is sufficient for this, to inactivate only one of these genes besides recA, which is why in a preferred use of this type, exactly one gene from phase IV of the sporulation is functionally inactivated.

In this way, safety strains for biotechnological production are obtained. They are less able to survive outside the optimal fermentation conditions, in particular under environmental conditions that include poor nutritional supply and DNA-harming factors, for example UV irradiation or aggressive chemicals. The first group of environmental factors would induce the gram-positive bacteria that are naturally capable of sporulation to convert into the permanent form of spores; the second group of factors can be counter balanced by microorganisms naturally over Reca-effected DNA repair processes and recombination processes. When the cells are incapable of both or even only severely limited by both, they are suitable according to the invention as safety strains.

It is further possible to inactivate, together with recA, one or more of the genes known from the prior art, thereby obtaining safety strains that besides being incapable of RecA-effected DNA recombination and forming mature spores are also characterized by these additional characteristics. For the production of GMOs, besides “active” systems that prevent viability and suitably stringent regulating systems, are included all those that have been designated as “passive” systems in the prior art illustrated in the introduction. They particularly include inactivating mutations in one or more of the following genes: epr, rp-I, rp-II, isp-1, apr, npr, spoOA, bpr, rsp, mpr, vpr, spo0A, spoII:D, spoIIAC, spo2, spo3, sigE, sigF, spoIIE, spoIISB, sigG, spoIVCB, spoIIIC, nprM and the gene for the isopropyl malate dehydrogenase (leuB). These gene identifiers are taken from the prior art illustrated in the introduction of the present application. Thus, these abbreviations used here are meant to refer to each of the described meanings in the applications and publications cited in the introduction. In the case that additional names have been established in the prior art for the same genes or gene groups encoding for the same proteins, particularly for the homologs in other species of bacteria, as those on which the cited publications are based, then the same equally applies.

However, according to the invention it is not absolutely necessary to inactivate a further gene in addition to recA and optionally an additional sporulation phase IV gene, so that preferably, additional mutations can be essentially dispensed with. The claimed advantage in the object of the present application is hereby linked with the establishment of the least possible parallel safety systems in the same cell. This requires a lower work effort than would be undertaken for the four different deletions as proposed in the cited work on B. megaterium. This is particularly relevant when the cells in question firstly—as long as they are still capable of recombination—are provided with transgenes relevant for the production and only then are converted into safety strains, in particular into a recA-minus-phenotype. These types of additional mutations are only indicated for very critical cases, for example highly pathogenic strains.

Due to the sequences made available by the present application, sporulation defects are produced in the genes spoIVA, spoIVB, spoIVCA, spoIVCB, spoIVFA, spoIVFB or yqfD in the nomenclature of B. subtilis or, in the case of Bacillus licheniformis in the gene spoIV or in the genes that are homologs to these, present in the particular host cells.

This homology can be developed as a first approximation through a sequence comparison. To check this, the gene in question can be inactivated in the microorganism strain provided for the biotechnological production and the functional agreement of the gene in question can be examined through a reproduction of the phenotype (rescue). If the parallel preparation of an inventively relevant spoIVA, spoIVB, spoIVCA, spoIVCB, spoIVFA, spoIVFB, yqfD- or spoIV-copy converts the knock-out mutant under consideration into a sporulation positive phenotype again, then this is the proof that a functional exchangeability of the genes under consideration also exists. According to the invention, genes that are homologous to the cited phase IV sporulation genes therefore particularly include those that are accessible by a “rescue” of this type. When possible it concerns a preferably used sporulation gene. Therefore, this control is particularly possible with reasonable effort because firstly, according to the invention, just one such functionally inactive mutant has to be produced and secondly, through the sequence protocol for the present application, the relevant sequences from B. subtilis and particularly preferred sequences thereof additionally from B. licheniformis are made available, over which a rescue of this type can be made.

In preferred embodiments, the inventive, previously described use for the functional inactivation of the genes spoIVA, spoIVB, spoIVCA, spoIVCB, spoIVFA, spoIVFB, yqfD or spoIV or of each of their homologous genes occurs with the help of the sequences SEQ ID NO. 3, 5, 7, 9, 11, 13, 15 or 17 or parts thereof, preferably with the help of parts that comprise at least 70 to 150 contiguous nucleic acid positions, particularly preferably with the help of two such parts that surround a part of the gene located between them.

As described above, these precise sequences, in particular for B. licheniformis und B. subtilis and closely related species, can be used to prepare suitable molecular biological constructs. For this, all of the possibilities for recA listed above are available and are correspondingly preferred.

Microorganisms obtained with the described process represent a separate embodiment of the present invention. In its broadest sense it therefore concerns a gram-positive bacterium that is not Bacillus megaterium in which the gene recA is functionally inactivated.

This mainly concerns gram-positive bacteria in which the gene recA has been functionally inactivated by genetic, i.e. synthetic methods.

As already stated, gram-positive bacteria, because for example of their ability to secrete products of value and/or their comparative ease of fermentation, are the most important microorganisms for biotechnology. Among these, different species are preferred for the various fields of application; low molecular weight compounds such as for example amino acids are produced to a large extent by means of corynebacteria; Bacillus and among these particularly B. licheniformis is particularly valued for the production of extra-cellular proteins. According to the invention, they are all accessible, at least in principle by a functional inactivation of RecA.

These inventive bacteria are characterized by the described recombination defects and therefore possess disadvantages with regard to their viability under natural conditions, particularly in competition with other microorganisms, and consequently are suitable as safety strains for the biotechnological production. This does not concern Bacillus megaterium for the reasons described above.

In accordance with the previous embodiments, those gram-positive bacteria are preferred for which the functional inactivation is effected through point mutagenesis, partial deletion or insertion or total deletion of the encoding region for the complete protein.

In accordance with the previous embodiments, those gram-positive bacteria are further preferred for which the functional inactivation is effected through an inventive nucleic acid that encodes for RecA and/or through a nucleic acid whose nucleotide sequence matches with the nucleotide sequence listed in SEQ ID NO. 31 in the positions 369 to 1415 to at least 1045, preferably at least 1046, quite particularly preferably 1047 of these 1047 positions or through the at least partially non-encoding flanking regions of these nucleic acids.

In this context, nucleic acids of the above described homology values to SEQ ID NO. 1 or, as already mentioned, to SEQ ID NO. 31 are correspondingly preferred In this regard, microorganisms in which the functional inactivation results from the nucleic acids or segments listed in SEQ ID NO. 1 or 31, represent the most preferred microorganisms.

In accordance with the above statements, in the case of a mutagenesis through crossing over, preferably boundary sequences of at least 70 to 150 bp each are used, and which can be checked through a sequencing of the chromosomal segments under consideration.

In accordance with the previous embodiments, those gram-positive bacteria and their genera Clostridium or Bacillus are further preferred, which are naturally capable of sporulation and in which at the same time a gene from the phase IV of the sporulation is functionally inactivated with recA.

In accordance with the above statements, hereafter, such gene defects are particularly understood to mean those that have been carried out through biotechnological interventions.

In accordance with the previous embodiments, those gram-positive bacteria are further preferred in which the inactivated gene from the phase IV sporulation in the nomenclature of B. subtilis concerns one of the genes spoIVA, spoIVB, spoIVCA, spoIVCB, spoIVFA, spoIVFB or yqfD or is a homologous gene to this, preferably in the case of B. subtilis is the gene yqfD, in the case of Bacillus licheniformis is the gene spoIV and all other cases is a homologous gene to this.

In particular cases, for example when using highly pathogenic strains, a plurality of the cited sporulation genes or one or more of the genes or groups of genes described in the prior art spoIV/yqfD/homolog, epr, rp-I, rp-II, isp-1, apr, npr, spoOA, bpr, rsp, mpr, vpr, spo0A, spoII:D, spoIIAC, spo2, spo3, sigE, sigF, spoIIE, spoIISB, sigG, spoIVCB, spoIIIC, nprM and/or the gene for the isopropyl malate dehydrogenase (leuB) can be functionally inactivated. These abbreviations are understood to have the meanings of those described in the applications and publications cited in the introduction, possible synonyms also being included.

However in accordance with the previous embodiments, such a gram-positive bacterium is preferred in which—besides the recA inactivation—exactly one gene from the phase IV sporulation is functionally inactivated.

In accordance with the above statement, such a gram-positive bacterium is further preferred in which the functional inactivation of the genes spoIVB, spoIVCA, spoIVCB, spoIVFA, spoIVFB, yqfD or spoIV or of each of their homologous genes occurs with the help of the sequences SEQ ID NO. 3, 5, 7, 9, 11, 13, 15 or 17 or parts thereof, preferably with the help of parts that comprise at least 70 to 150 contiguous nucleic acid positions, particularly preferably with the help of two such parts that surround a part of the gene located between them.

This can be verified through preparations of the DNA in question, for example the chromosomal DNA of an inventive strain, and restriction analysis or PCR. Each of the flanking sequences can be used as the primer for this, wherein the size of the PCR product provides information about the presence and possibly the size of the insert. This method is illustrated for spoIV in the examples of the present application.

In accordance with the previous embodiments, particularly preferred inventive gram-positive bacteria include those which concern a representative of the genera Clostridium or Bacillus, in particular those of the species Bacillus subtilis, B. licheniformis, B. amyloliquefaciens, B. stearothermophilus, B. globigii, B. clausii or B. lentus, and quite particularly strains of B. licheniformis.

The methods for fermenting an inventive gram-positive bacterium represent a separate subject matter of the invention.

These methods are characterized in that RecA, preferably in combination with the illustrated preferred embodiments, is not active and the strain in question represents a significantly minimized safety risk in the case of an accidental release from the unit into the environment. Fermentation methods are subject to suitable safety requirements such that they may be operated only when these requirements are met.

That such a strain is not fundamentally handicapped under the optimal conditions during fermentation is proved by example 5 (FIG. 6) of the present application; this is also true for the double mutant described there. However, the inactivation of recA leads to a significantly reduced viability under the effect of UV. This is a normal environmental factor that would confront the bacteria should they possibly exit the production unit into the surroundings. In addition, UV irradiation is a normal method of sterilization in laboratories and biotechnological production factories. As is further proved by the examples, the inactivation of the spoIV leads to a drastically reduced sporulation rate. As a result of these examples, both factors can be combined with each other in the same bacterial strain. Moreover, due to their different fundamental methods of action, both “passive” systems complement each other for the production of industrially useful safety strains.

Preferably, these methods concern the manufacture of a product of value, in particular a low molecular weight compound or a protein.

Indeed, it is also advantageous if suitable strains are also employed in a lab scale. Here, however, it is generally easier to fulfill the typical general conditions. Moreover, the major application of fermentation of microorganisms consists in the biotechnological manufacture of products of value.

In accordance with the importance of these materials, such methods are preferred in which the low molecular weight compound concerns a natural product, a nutritional supplement or a pharmaceutically relevant compound.

These include, for example, amino acids or vitamins, which are particularly used as nutritional supplements. The pharmaceutically relevant compounds concern precursors or intermediates for medicaments or even the medicaments themselves. All these cases concern biotransformation, in which the metabolic properties of the microorganisms are exploited in order to totally replace or at least replace some of the steps of the otherwise laborious chemical syntheses.

In no less preferred methods, the protein concerns an enzyme, in particular an enzyme from the group of the α-amylases, proteases, cellulases, lipases, oxidoreductases, peroxidases, laccases, oxidases and hemicellulases.

Industrial enzymes that are manufactured in this type of process are used, for example, in the food industry. Thus, α-amylases serve, for example, to prevent bread going stale or for clarifying fruit juices. Proteases are used to decompose proteins. All these enzymes are described for use in detergents and cleansing agents, wherein the subtilisin proteases in particular, already produced naturally from gram-positive bacteria, take a prominent place. They are particularly used in the textile and leather industry for reconditioning natural products. Moreover, all these enzymes can be employed, once again in the context of biotransformation, as catalysts for chemical reactions.

As initially stated in the object of the invention, it was desired to find a safety system of this type that is not so specific as to prevent it also being used in other molecular biological approaches. These types of approaches can be summarized in a further independent subject matter of the invention.

Generally speaking, this means the use, in a molecular biological reaction approach, of the above-described factor RecA and/or a RecA that matches with the amino acid sequence listed in SEQ ID NO. 32 in at least 347, preferably 348 of the 348 amino acid positions shown there.

To this end, its naturally available activities are exploited.

The use is consequently preferred for stabilizing single strand DNA, particularly in a DNA polymerization, for recombination processes in vitro, or for converting double stranded DNA into single stranded DNA or vice versa.

RecA is a DNA single stranded protein that, as mentioned, also exhibits a certain affinity to double stranded DNA. This function has an effect during the natural process of crossing over in the course of homologous recombination. Thus, RecA can be added, for example, to a PCR or a preparation of DNA phages in order to stabilize the single strands. When in vitro recombination processes are reproduced, for example by introducing mutations (thus also for the inventive mutations listed above), then they can be facilitated by RecA. Finally, the conversion of double stranded DNA into single stranded DNA or vice versa is understood to mean a gyrase or gyrase supporting function. This can be exploited to influence the DNA topology, for example in work with plasmid DNA.

Vectors that comprise a previously described nucleic acid represent a separate subject matter of the invention. The present invention is also realized in this form. Thus, this DNA can be molecular biologically treated or stored in the form of cloning vectors.

Preferably, such a vector is an expression vector. It can be utilized to produce an inventive RecA and to convey the cited applications of the factor.

Accordingly, methods for manufacturing a previously described factor RecA also represent a separate subject matter of the invention.

This preferably concerns methods that occur using one of the above described nucleic acids, in particular those with increasing homology values to SEQ ID NO. 1, preferably a corresponding expression vector and further preferably by fermentation of a host comprising this nucleic acid or these expression vectors.

Thus, the present invention is realized in that a cell acquires and translates such a gene in the form of a chromosomal copy. On the other hand, the provision of this gene seems more easily controllable in the form of a plasmid, which optionally provides a plurality of copies for the formation of this factor.

The use of the inventive nucleic acid that encodes a factor RecA to express this factor represents a separate subject matter of the invention. In accordance with the above statements, the present invention is realized in at least one aspect.

Preferably this serves to produce this factor itself, particularly in one of the processes described above. Alternatively, the intracellular expression can also serve to modulate molecular biological activities of the cells in question, in particular in recombination processes in vivo.

The inactivation, for example by an antisense-approach or RNA interference approach, is meant here, according to which the mRNA encoding for RecA is selectively switched off or is rendered only partially translatable. In this way the expression of this factor can be selectively modulated quite successfully. This is valid both for biotechnological production strains as well as for laboratory approaches for studying molecular biological aspects.

Furthermore, the present invention is also realized by the use of the above-described inventive nucleic acid that encodes for a Reca factor and/or a nucleic acid that encodes for a Reca factor whose nucleotide sequence coincides with the nucleotide sequence listed in SEQ ID NO. 31 in positions 369 to 1415 to at least 1045, preferably at least 1046, particularly preferably 1047 of these positions, for the inactivation of this factor or the gene recA in an in vitro approach, in particular through interaction with an associated nucleic acid.

This can be advantageous for preventing recombination processes, particularly for in vitro transcription or translation approaches.

Additional embodiments of the present invention stem from the molecular biological viewpoint through which the recA and spoIV genes are accessible. As illustrated in example 1, the associated DNA segments could be obtained through PCR with the help of the oligonucleotides listed in SEQ ID NO. 19 to 30 from principally any B. licheniformis strain, such that the present invention can be particularly easily reproduced.

Consequently, an embodiment of the present invention is a remote nucleic acid encoding for a partial sequence of recA or for a neighboring partial sequence with recA in vivo, of preferably less than 1000 bp, particularly preferably less than 500 bp according to one of the SEQ ID NO. 25 to 30.

They are partial sequences of recA or those that are remote from recA by only some hundred intermediate base pairs. Increasingly preferred are 1000, 900, 800, 700, 600, 500, 400, 300, 200, 100 bp up to an immediate vicinity, i.e. a location at the beginning or at the end of recA, preferably in the areas that are still not yet encoding for proteins. According to example 1 for the extraction of a recA, they can be obtained from a not further characterized strain, wherein the chance of success increases with increasing similarity to B. licheniformis.

In accordance with the previous embodiments and illustrated by example 1, a use of at least one, preferably of at least two nucleic acids orientated towards one another according to SEQ ID NO. 25 to 30 for the amplification of an in vivo DNA region enclosed thereby also corresponds to the present invention.

The paired usage results from the PCR approach that fundamentally requires primers opposite to one another. The orientation of the primers in question is shown in FIG. 3B. Due to the comparatively high homology values, in principle it can be assumed that these primers also assume a similar orientation in not yet characterized recA genes.

A preferred possibility of use consists in firstly producing the furthest externally binding primer (for example recA6 in combination with recA5 according to FIG. 3B or, if this does not work, recA1 and/or recA4), in order to obtain the intermediate located region. Then, to produce the concrete deletion constructs, further internally binding oligonucleotides can be employed as the primer, for example recA2 (for example in combination with recA2) and recA3 (for example in combination with recA4), wherein the nucleotide sequences of the internal primer can be corrected if necessary with the sequences resulting from the preceding PCR. Should this method fail, PCR primers with sequence variations can also be employed, as is known per se and has already been stated above. The success of this approach authenticates the sequencing of the obtained fragments that should exhibit significant homologies to those sequences listed in SEQ ID NO. 1 and/or 31 or in FIG. 2, when they are, as desired, a recA gene.

This preferably concerns a use for the amplification of a recA gene, as in this way the aspect of the inactivation of this gene is realizable according to the present invention.

Accordingly, this also preferably concerns such uses in the scope of a process described above in detail for the functional inactivation of the gene recA in a gram-positive bacterium that is not Bacillus megaterium, including the above-described embodiments of this aspect of the invention.

Analogously, this also preferably concerns such uses for the production of a gram-positive bacterium that is not Bacillus megaterium, in which the gene recA is functionally inactivated, including the above-described embodiments of this aspect of the invention.

Further aspects of the present invention are characterized by the inactivation of the gene spoIV. In accordance with the last explanations to recA, the following aspects also concern the realizations of the present invention:

A remote nucleic acid encoding for a partial sequence of spoIV or for a neighboring partial sequence with spoIV in vivo, of preferably less than 1000 bp, particularly preferably less than 500 bp according to one of the SEQ ID NO. 19 to 24;

A use of at least one, preferably at least two nucleic acids orientated towards one another according to SEQ ID NO. 25 to 30 for the amplification of an in vivo DNA region enclosed thereby;

such a use for the amplification of a spoIV gene;

such a use in the scope of a process for the functional inactivation of the gene recA in a gram-positive bacterium that is not Bacillus megaterium, wherein simultaneously with recA a gene from phase IV of the sporulation is functionally inactivated, including the above-described embodiments of this aspect of the invention;

such a use for the production of a gram-positive bacterium that is not Bacillus megaterium, preferably one of the genera Clostridium or Bacillus that is naturally capable of sporulation and wherein simultaneously with recA a gene from phase IV of the sporulation is functionally inactivated, including the above-described embodiments of this aspect of the invention;

### EXAMPLES

All molecular biological steps follow standard methods, as are illustrated, for example in the handbook from Fritsch, Sambrook und Maniatis “Molecular cloning: a laboratory manual”, Cold Spring Harbour Laboratory Press, New York, 1989 or “Mikrobiologische Methoden: Eine Einführung in grundlegende Arbeitstechniken” (“Microbiological Methods: An introduction into fundamental techniques”) by E.Bast (1999) Spektrum Akademischer Verlag, Heidelberg, Berlin, or comparable pertinent works Enzymes and kits were used according to the directions of the relevant manufacturer.

### Example 1

Isolation of the spoIV- and the recA-Region from a B. licheniformis Laboratory Strain

Fundamentally, the present invention can be implemented starting with the sequences for recA and spoIV from B. licheniformis DSM 13 (SEQ ID NO. 1 or 3), listed in the sequence printouts. Here, it was started even earlier by firstly employing a PCR-based process to isolate this gene from a Bacillus strain, as is described in the publication “A general method for cloning recA genes of Gram-positive bacteria by polymerase chain reaction” (1992) by Duwat et al. in J. Bacteriol., vol 174 (Nr. 15), pp. 5171-5175.

For this, the PCR primers were synthesized from the already known DNA sequences of the genes spoIV und recA from various gram positive bacteria and particularly from B. licheniformis DSM 13, of which the finally successful ones are listed in Table 1 and in the sequence printout of the present application. Their binding loci on the respective gene loci are presented in FIG. 3.

TABLE 1
Oligonucleotides used for amplifying the spolV-
and the recA-locus.

| | | |
|---|---|---|
| Name | SEQ ID NO | 5′-3′ Sequence |

| | | |
|---|---|---|
| spo1 | 19 | GGCTGATGCTCAAACAGGGGCAGTGCATC |
| spo2 | 20 | CATGAACGGCCTTTACGACAGCCA |
| spo3 | 21 | GTCATCAAAACGATTTTGCCTGAGG |
| spo4 | 22 | ATGTTCTGTCCCGGGATTGGCTCCTG |
| spo6 | 23 | GTTTTGACTCTGATCGGAATTCTTTGGCG |
| spo7 | 24 | GCACGAAACGAGCGAGAATGGC |
| recA1 | 25 | GGAATTCGGCATCAGCTTCACTGGAG |
| recA2 | 26 | GCTATGTCGACTATACCTTGTTTATGCGG |
| recA3 | 27 | GACCTCGGAACAGAGCTTGAC |
| recA4 | 28 | TCAAACTGCAGTCATTAAGAGAATGGATGG |
| recA5 | 29 | AAGCTTACGGTTTAACGTTTCTG |
| recA6 | 30 | ACACAAACGAATTGAAAGTGTCAGCG |

In this way, overlapping parts of both the spoIV- and the recA-locus were isolated from a preparation of chromosomal DNA of a B. licheniformis laboratory strain by means of PCR techniques. This strain, designated as B. licheniformis, served as the example for any gram-positive bacterium. This approach can be used in principle for all gram-positive bacteria, particularly since these primers are now known.

After sequencing the PCR fragments resulting from standard PCR, they were each assembled to a total sequence. In the case of the spoIV region, this matched 100% over the total length of 1792 bp with the sequence from B. licheniformis DSM 13 listed in the sequence printout SEQ ID NO. 3. Therefore, the species designation found in field <213> does not designate the specific strain DSM 13 or A but rather the species in general.

Furthermore, if one adds to this sequence, such that the encoding region includes the positions shown as 140 to 1336 (including the stop codon), the first three encoding for the start codon GTG that is translated in vivo as methionine. The total segment shown from 1792 bp is designated here as the gene spoIV, because it does not solely comprise the protein encoding part but also regulatory elements that relate to this gene. This gene designation also follows notwithstanding the fact that segments that are primarily assigned to other genes may possibly extend inside. This appears justifiable because gene regions are sometimes found to be overlapping.

In the case of the recA-region, a DNA was obtained with a length of 1557 bp, which matches with the homologizable, directly protein-encoding region in SEQ ID NO. 1 of the present application in all except three positions. It is shown in SEQ ID NO. 31. The derived amino acid sequence can be found in SEQ ID NO. 32. The differences to SEQ ID NO. 1 on the DNA level are in the positions 282-284, by which the associated codons encode for EH instead of the amino acid sequence DT. Due to this difference, the species designations to SEQ ID NO. 1 and 31 were supplemented in the field <213> with the strain designations DSM 13 and A. It should be added that the encoding region includes the positions 369 to 1415 (including the stop codon) shown in SEQ ID NO. 31. In accordance with the discussions on spoIV, the total segment shown of 1557 bp is designated as the gene recA.

Both loci spoIV and recA obtained in this way were deposited in the databank GenBank (National Center for Biotechnology Information NCBI, National Institutes of Health, Bethesda, Md., USA) under the registration numbers AJ616332 (for spoIV) and AJ511368 (for recA).

### Example 2

Deletion of the spoIV- and the recA-Gene by Selective Gene Disruption

By the use of the selective gene disruption technique, a larger possible region could be deleted from the spoIV- or the recA gene. The experimental design is sketched out in FIG. 3. Part A shows the introduction of the deletion in spoIV and thus the derivation of the strain B. licheniformis A.1 (ΔspoIV) from B. licheniformis A. Part B shows the further development of B. licheniformis A.1 (ΔspoIV) to B. licheniformis A.2 (ΔspoIV, ΔrecA). The gene locus under consideration, including each of the directly flanking genes is also designated as are important restriction cutting sites and the binding regions for the primers listed in example 1.

For the deletions, flanking regions from the chromosomal DNA were amplified with the oligonucleotides shown in FIG. 3 and used for the construction of suitable deletion cartridges, as is described below in more detail. They were created first in the E. coli vector pUCBM21. This is described under http://seq.yeastgenome.org/vectordb/vector_descrip/PUCBM21.html (accepted on 14.1.2005) and is commercially available from Roche Diagnostics GmbH, Roche Applied Science, Sandhofer Str. 116, 68305 Mannheim (formally Boehringer). They were later cloned into the Bacillus vector pE194. This is described under http://seq.yeastgenome.org/vectordb/vector_descrip/PE194.html (accepted on 14.1.2005) and is available from the American Type Culture Collection, 10801 University Boulevard, Manassas, Va. 20110-2209, USA (http://www.atcc.org).

The production of a gene disruption by means of such vectors occurs during recombination events through the corresponding homologous flanking regions. Here, the original plasmid-localized, in vitro mutated copy of the gene under disruption is exchanged against the native, intact copy in the bacterial chromosome by means of two successive single crossover events. As the Bacillus vector carries a temperature sensitive origin of replication, then after the successful disruption under non-permissive conditions (42° C.), the plasmid fractions can be removed again later from the cells, thereby permitting the establishment of a stable line of mutants.

The oligonucleotides spo3 and spo4, together with spo7 and spo6, were used to construct the spoIV deletion cartridge; both flanks are each ca. 450 bp large and frame a region of 740 bp (size of the later deletion). The oligonucleotides recA1 and recA2, together with recA3 and recA4, were used to construct the recA deletion cartridge; both flanks are each ca. 340 bp large and frame a region of 852 bp (size of the later deletion). After cloning the deletion cartridge into the singular Pstl cutting site of pE194 (carried out in B. subtilis DB104; strain described in Kawamura, F. and Doi, R. H. (1984), J. Bacteriol., vol. 160, pages 442-444), both the disruption vectors pESpo2 and pErecA2 were obtained. Then, for a selective deletion of the spoIV gene, the vector pESpo2 was transformed in B. licheniformis A via protoplast technique (described in S. Chang und S. N. Cohen, (1979) Molec. Gen. Genef., vol. 168, pages 111-115). Under non-permissive conditions, the vector was subsequently again thinned out of the cells from a suitable transformant line. At the same time the presence of a mutant amplification was investigated by PCR using the oligonucleotides spo1 and spo2, and a stable ΔspoIV mutant line (designated B. licheniformis A.1) was successfully isolated after several cultivation passages.

This mutant line A.1 was used for a further transformation with the recA disruption factor pErecA2. Analogously the transformant was also sub cultivated in several cultivation passages at 42° C., a corresponding ArecA mutant (ΔspoIV/ArecA double mutant, designated as the B. licheniformis A.2) being identified by means of a screening for mitomycin C-sensitivity (0.03 μg/μl). In addition, these phenotypical findings were verified by means of a PCR using the oligonucleotides recA6 and recA5.

### Example 3

Genotypical Characterization of the AspoIV Single Mutant and the ΔspoIV/ΔrecA Double Mutant

Both mutant strains (B. licheniformis A.1 und A.2) were examined in comparison with the starting strain B. licheniformis A at the DNA level in order to check the deletions (truncations) in the corresponding gene region. Thus, by means of the PCR technique using the promers spo1 and spo2, the 740 bp deletion could be clearly detected in the spoIV locus of the strains A.1 and A.2 (FIG. 4 A left part). The same is true for the 852 bp deletion in the recA gene of the mutant A.2, using the primer pair recA6 and recA5 (FIG. 4 A1 right side).

In addition, the three strains were subjected to a Southern analysis. The spoIV deletion was detected by cutting 2 μg of each chromosomal DNA with the restriction endonuclease C/al and after separation by gel electrophoresis they were hybridized using standard methods with a DIG-marked PCR product (generated with the primers spo3 and spoowith the starting DNA). The larger of the two detected ClaI fragments both for strain A.1 and also for A.2 appeared to be at a level lower than from the starting strain A corresponding to the size of the deletion (FIG. 4 B left side).

The recA deletion was detected by digesting the DNA with the restriction endonuclease Sspl and hybridized in an analogous manner with a DIG marked PCR product (generated in an analogous way with the primers recA1 and recA2). In this case, due to the deletion, the corresponding Sspl fragment for the strain A.2 was also at a correspondingly lower level than in the parental strain A.1 or the wild type strain A (FIG. 4 B right side).

### Example 4

Phenotypical Characterization of the ΔspoIV Single Mutant A.1 and the ΔspoIV/ΔrecA double mutant A.2: Survival rate and spore formation

The culture for the sporulation test was carried out in 200 ml of Schaffer's sporulation medium (16.0 g LB-Medium, 2.0 g KCl, 0.5 g MgSO₄, x 7H₂O, ad 993,0 ml dist. water; pH 7.0; the solution is autoclaved and then supplemented with the following components: 1 ml Ca(NO₃)₂ (0.1 M), 1 ml MnCl₂ (0.1 M), 1 ml FeSO₄, (1 mM), 4 ml Glucose (20% (w/v)), in 500 ml flasks equipped with two baffles. Three flasks of each of the three strains under test were inoculated with 0.25% of a LB preculture and incubated at 30° C. as well as ca. 120 rpm (Innova 4230, New Brunswick Scientific, Edison, N.Y., USA). Samples of 1100 μl culture were transferred into a sterile Eppendorf bottle. 100 μl of this aliquot were used to determine the living cell count, by dilution in 15 mM NaCl. Each of the dilution steps were plated onto four LB agar plates and incubated overnight at 30° C. The number of viable cells was determined by counting the colonies on each of the four agar plates. The living cell count [colony forming units (cfu)] was determined by considering the plated volume and the dilution step and the values of a series of plates were averaged. The remaining 1000 μl samples were incubated at 80° C. for 30 minutes in the water bath. 250 μl of the treated suspension were plated onto four LB agar plates and incubated at 30° C. The spore titer was determined by counting the germinated spores that form a single colony. The spore count of the plates was averaged and then calculated per ml culture. The results are illustrated in Table 2 and FIG. 5.

TABLE 2
Average values of the living cell count and the surviving
spores per ml culture.
Each strain was examined in three parallel experiments (= cultures).
Each experiment was statistically validated by four determinations.

| | A | | A.1 | | A.2 | |
|---|---|---|---|---|---|---|
| | Living cell | | Living cell | | Living cell | |
| Time | count | spores/ | count | spores/ | count | spores/ |
| [h] | [cfu/ml] | ml | [cfu/ml] | ml | [cfu/ml] | ml |
| 1 | 1.9E+04 | 0 | 2.6E+04 | 0 | 2.3E+04 | 0 |
| 3 | — | 0 | — | 0 | — | 0 |
| 6 | 1.7E+05 | 0 | 6.5E+04 | 0 | 1.7E+05 | 0 |
| 9 | 2.3E+06 | 0 | 1.2E+05 | 0 | 1.2E+06 | 0 |
| 12 | 3.6E+06 | 0 | 2.3E+05 | 0 | 2.3E+07 | 0 |
| 24 | 2.6E+07 | 0 | 1.6E+07 | 0 | 9.6E+07 | 0 |
| 36 | 2.7E+08 | 0.125 | 3.6E+08 | 0 | 3.6E+08 | 0 |
| 48 | — | 1.375 | — | 0 | — | 0 |
| 72 | 1.1E+09 | 13.5 | 1.6E+09 | 0 | 2.7E+09 | 0 |
| 96 | 2.6E+09 | 18.25 | 1.8E+09 | 0 | 3.9E+09 | 0 |
| 168 | 2.2E+09 | 33.1 | 1.5E+09 | 0 | 1.3E+09 | 0 |
| 216 | 6.7E+06 | 35 | 6.5E+05 | 0 | 9.4E+06 | 0 |
| 264 | 5.5E+05 | 36 | 10 | 0 | 3.7E+03 | 0 |
| 336 | — | 34 | — | 0 | — | 0 |

A = starting strain B. licheniformis A;
A.1 = B. licheniformis A.1 (ΔspoIV);
A.2 = B. licheniformis MD1.2 (ΔspoIV, ΔrecA).

From these results, one sees that only the cells of the starting strain are capable of spore formation. Due to the deletion of spoIV, both the mutants are incapable of spore formation. The mutant characterized by the additional deletion of the recA gene exhibits a somewhat less steep decrease in the living cell count.

### Example 5

Growth Curves of the ΔspoIV Single Mutant A.1 and the ΔspoIV/ΔrecA Double Mutant A.2

For this test, 10 ml cultures of the three previously described strains were each first inoculated with a single colony (from LB plate) and incubated at 37° C. and 150 rpm overnight. 50 ml minimal medium according to Sambrook et al. 1989 (1% (w/v) glucose, 0.1 mM CaCl₂, 0.01% (w/v) yeast extract, 0.02% (w/v) Casamino acids; pH 7.0) were each inoculated with 2% of these precultures in 250 ml Erlenmeyer flasks equipped with two baffles. These cultures were cultured at 37° C. and 180 rpm (shaker Innova 4230, New Brunswick Scientific, Edison, N.Y., USA) to reach an OD₅₄₆ of ca. 1.0 (late logarithmic growth phase).

The resulting growth curve up to the exponential growth phase is illustrated in FIG. 6. One notes that all three strains demonstrate practically the same results with regard to their doubling rate. A change from one strain to the others is therefore not associated with any detectable growth inhibition.

### Example 6

Phenotypical Characterization of the ΔspoIV Single Mutant A.1 and the ΔspoIV/ΔrecA Double Mutant A.2: UV Sensitivity

In connection with the previous example, the cells at the time of the logarithmic growth phase in FIG. 6 were quantitatively tested for their UV sensitivity by dilution with 15 mM NaCl solution to a level of 10⁻⁴ and plating 100 μl aliquots of each of the dilutions onto LB plates. Excepting two LB plates of each that served later as control plates, the remaining LB plates were then irradiated for different times with UV light at a wavelength of 254 nm: the plates were placed under a UV lamp with a power of 100 μW/cm², covered up after different irradiation times and wrapped in aluminum foil for protection against further light. The different irradiation times of 2 to 60 seconds corresponded to UV irradiation intensities of 2 to 60 J/m at the abovementioned power of the lamp. Control plates and UV plates were then incubated for 16 hours in the dark at 37° C. Based on the colony counts of the control plates, whose values correspond to a survival rate of 100%, and on the UV plates, the percentage survival rates were calculated as a function of the UV irradiation intensity. The double determinations from a total of three tests (=cultures) were averaged (see Table 3) and presented in the form of a graph (FIG. 7A).

TABLE 3
Averaged percentage survival rates of strains A, A.1 and
A.2 as a function of the UV irradiation intensity.
Each strain was examined in three experiments (= cultures). Each
experiment was statistically validated by double determination.

| | |
|---|---|
| | Percentage |
| UV irradiation intensity | survival rate (%) |

| (J/m²) | A | A.1 | A.2 |
|---|---|---|---|
| 0 | 100 | 100 | 100 |
| 2 | — | — | 7.73 |
| 4 | — | — | 3.28 |
| 6 | — | — | 0.87 |
| 8 | — | — | 0.28 |
| 10 | 46.7 | 43.1 | 0.01 |
| 20 | 18.73 | 18.85 | 0.01 |
| 30 | 6.27 | 6.02 | 0.01 |
| 40 | 1.95 | 2.08 | 0.01 |
| 50 | 0.33 | 0.36 | 0.01 |
| 60 | 0.03 | 0.13 | 0.01 |

A = B. licheniformis A;
A.1 = B. licheniformis A.1 (ΔspoIV);
A.2 = B. licheniformis A.2 (ΔspoIV, ΔrecA).

For a qualitative comparison of both the strains A.1 and A.2 in regard to their UV sensitivity, 15 μl aliquots from parallel cultures were each exposed on four LB plates. The plates were subsequently covered on their right side with a Plexiglas slide and irradiated with UV light on the other half (the left side). Then they were incubated as above for 16 hours at 37° C. in the dark.

The result of this experiment is shown in FIG. 7B. One notes that the investigated double mutant is considerably more UV sensitive than the simple mutant. Both parts of the experiment therefore prove that the deletion of recA, particularly in combination with spoIV, leads to mutants that are incapable of survival under the effects of natural environmental conditions. This effect, as illustrated in the specification, can be exploited for the use of these mutants as safety strains.

### DESCRIPTION OF THE FIGURES

FIG. 1: Amino acid sequence alignment of SEQ ID NO. 2 with closest prior art Rec factors.

The following meanings apply:

1: Factor RecA from B. licheniformis DSM 13 (SEQ ID NO. 2)
2: Factor RecA from B. amyloliquefaciens (AJ515542 in NCBI)
3: Factor RecA from B. subtilis (Z99112 in NCBI; Region 161035 to 162078)
4: Factor RecE from B. subtilis (X521 32 in NCBI)

FIG. 2: Nucleic acid sequence alignment of SEQ ID NO. 1 with closest prior art rec genes.

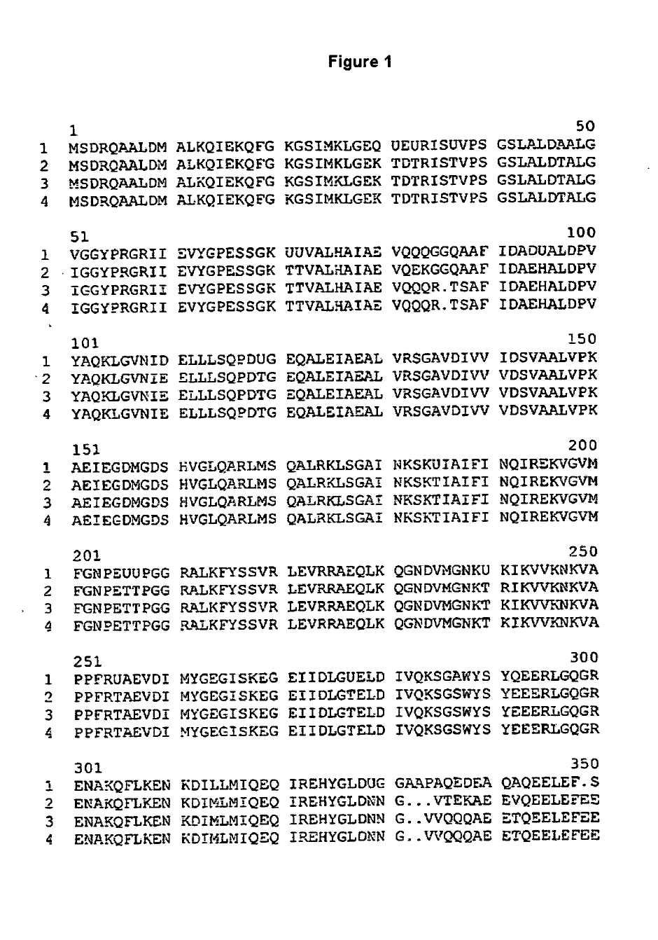

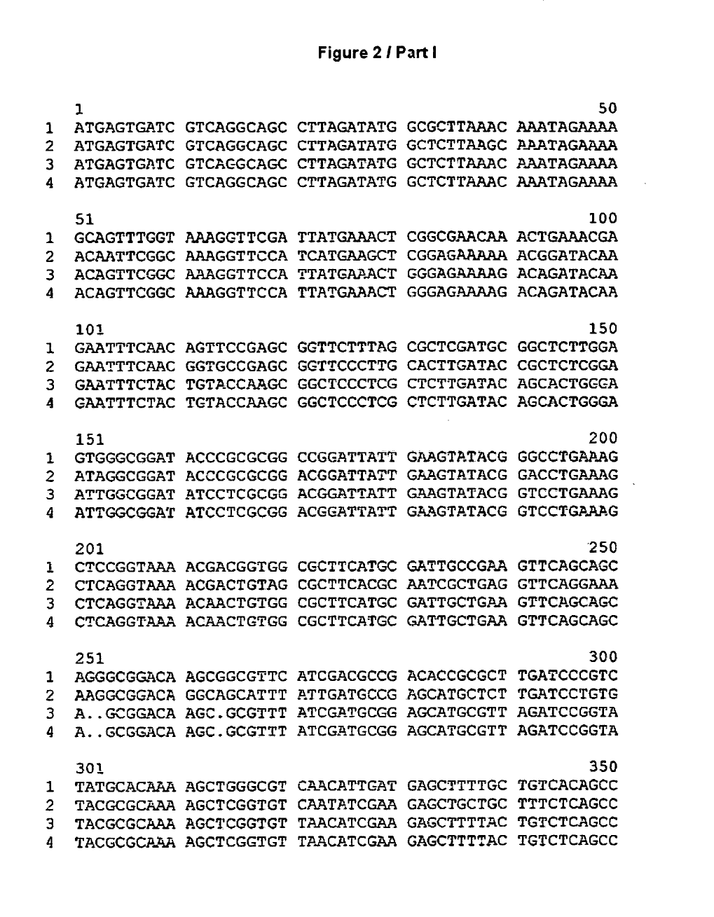

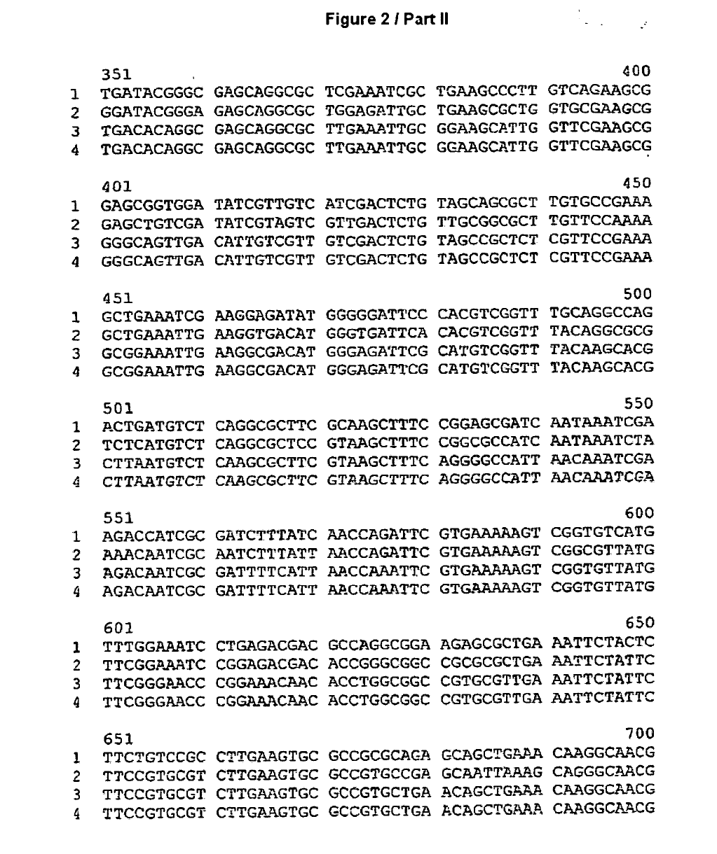

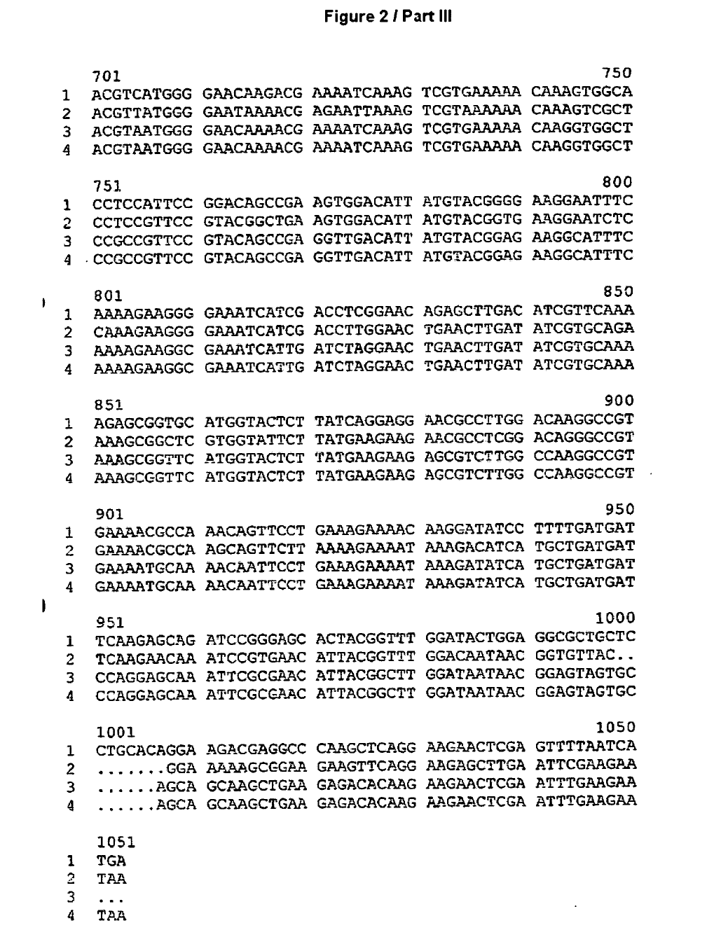

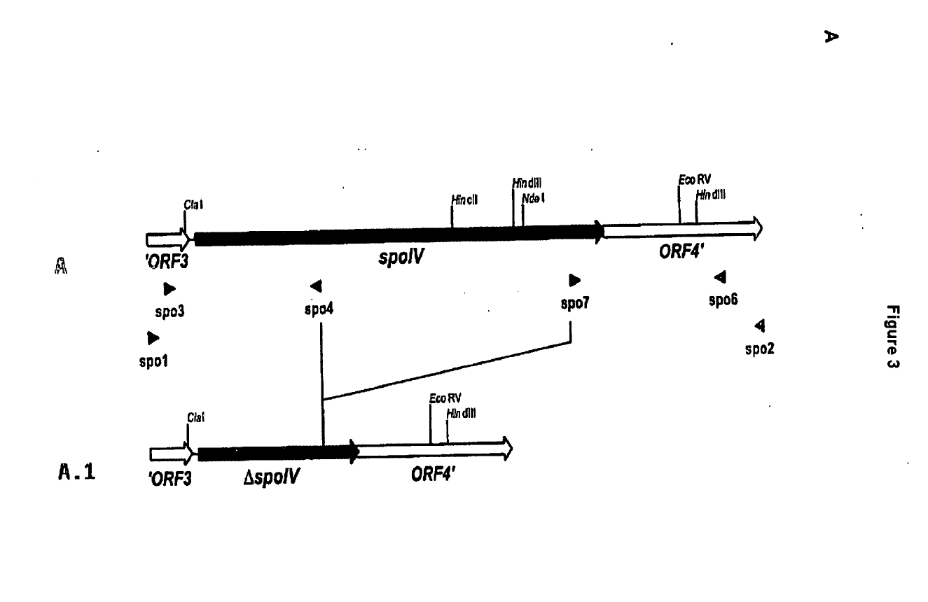

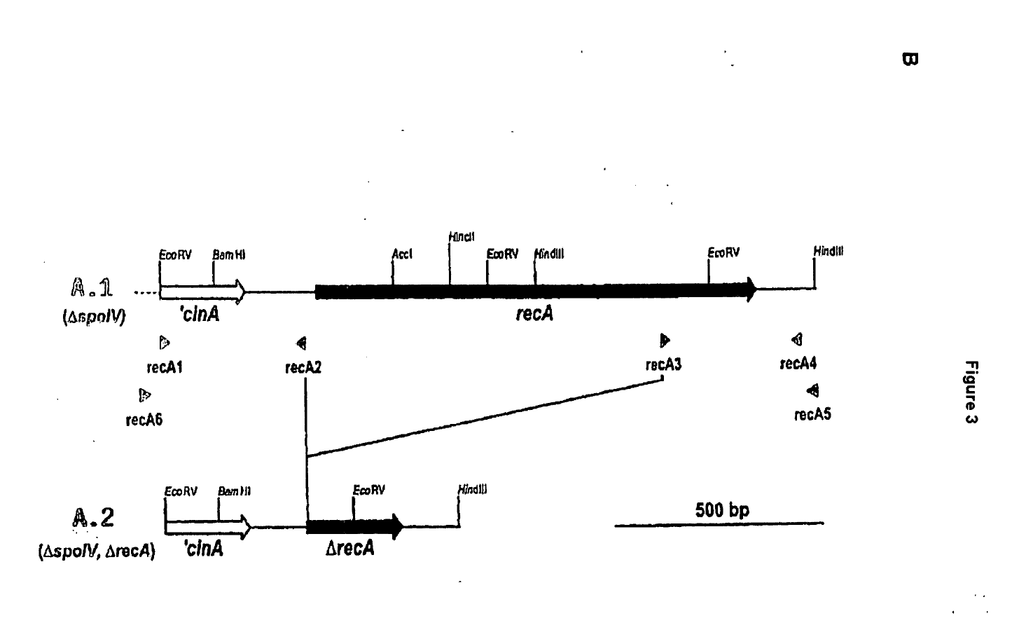

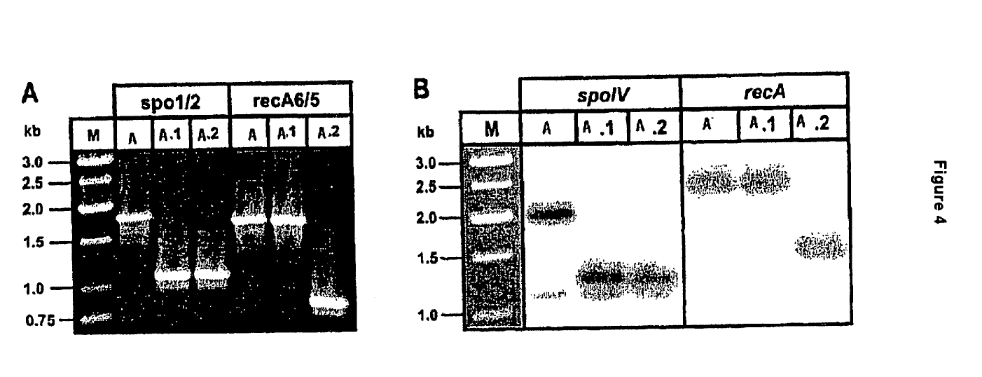

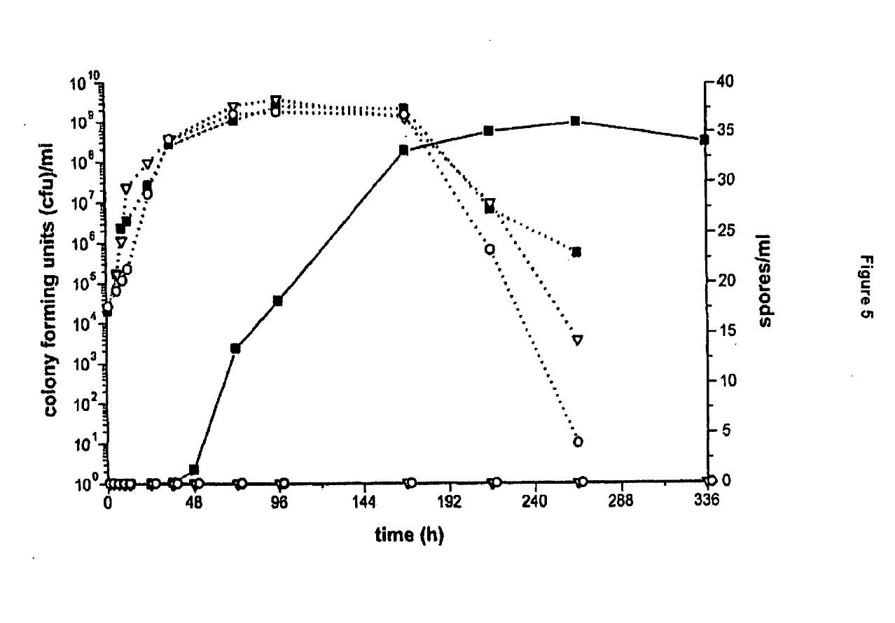

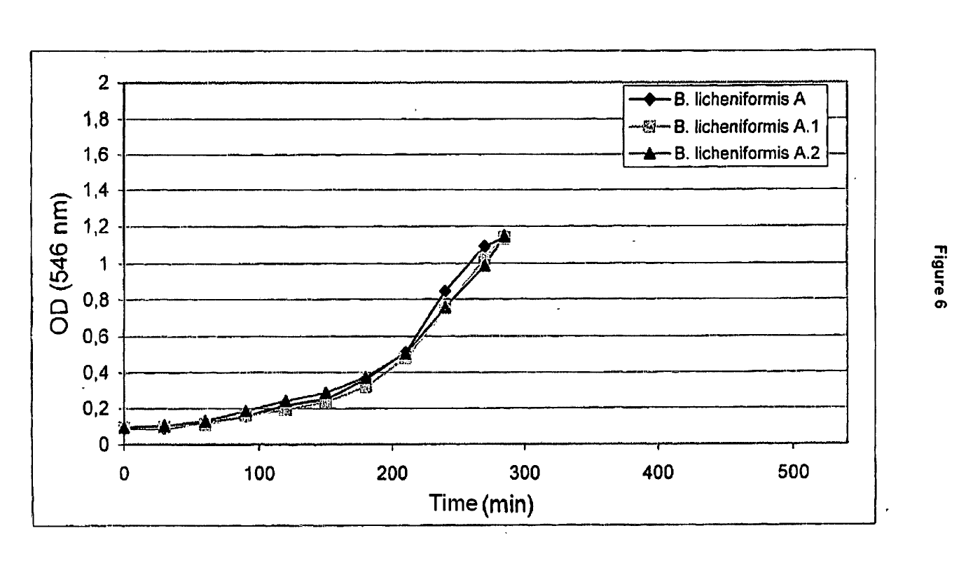

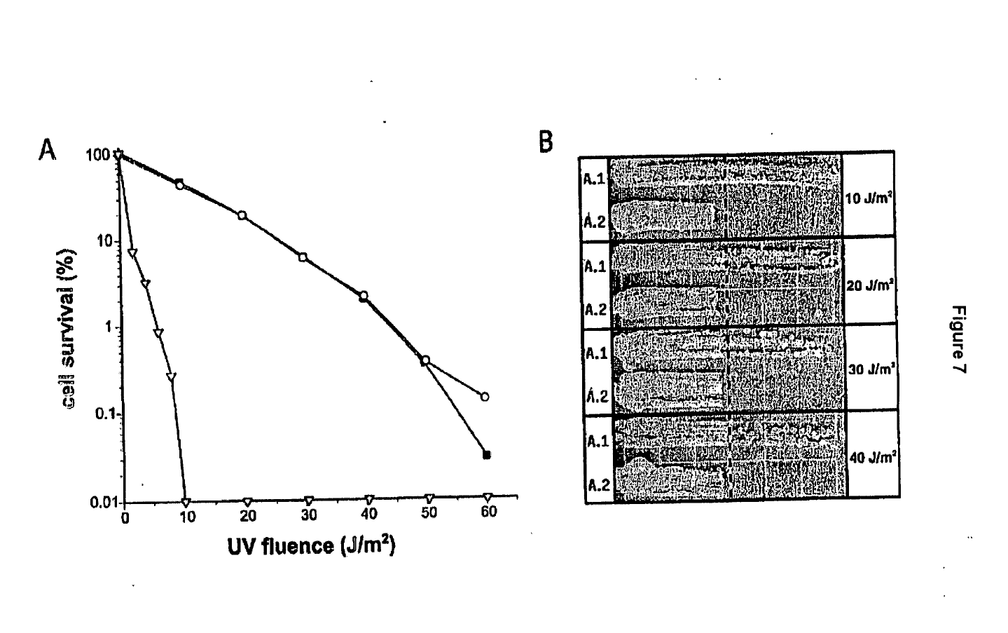

The following meanings apply:

1: Gene recA from B. lichenifomis DSM 13 (SEQ ID NO. 1)
2: Gene recA from B. amyloliquefaciens (AJ515542 in NCBI)
3: Gene recA from B. subtilis (Z99112 in NCBI; Region 161035 to 162078)
4: Gene recE from B. subtilis (X521 32 in NCBI)

FIG. 3: Schematic Representation of the genetic organizations of the wild type as well as of the mutant-loci of spoIV (A) and recA (B), including the binding points for the primers listed under SEQ ID NO. 19 to 30.
A Functional inactivation (deletion) of spoIV, i.e. derivation of the strain B. licheniformis A.1 from B. licheniformis A (see example 2).
B Functional inactivation (deletion) of recA, i.e. derivation of the strain B. licheniformis A.2 from B. licheniformis A.1 (see example 2).

FIG. 4: Genotypical investigation of the mutant strains A.1 and A.2 compared with the starting strain B. licheniformis A by means of PCR (A) and Southern analysis (B) (see example 3).

FIG. 5: Graph of the living cell counts and the spore titer of the B. licheniformis-cultures. Each culture was examined in three parallel experiments. Each experiment was statistically validated by four determinations (see example 4).

The following meanings apply:

Black, solid square: B. licheniformis A;
open circle: B. licheniformis A.1 (ΔspoIV);
open triangle: B. licheniformis A.2 (ΔspoIV, ArecA);
dashed line: Living cell counts
solid line: spore titer

FIG. 6: Growth curve of a culture of three B. licheniformis strains in minimal medium (see example 5).

FIG. 7: Results of the UV tests
A Graph of the survival rates after UV irradiation. Each culture was examined in three parallel experiments. Each experiment was statistically validated by double determinations (see example 6).
   The following meanings apply:
Black, solid square: B. licheniformis A;
open circle: B. licheniformis A.1 (ΔspoIV)
open triangle: B. licheniformis A.2 (ΔspoIV, ArecA).
B Qualitative UV test with exposure of the strains A.1 and A.2 on plates, which were half covered during the irradiation.

1. Factor RecA with an amino acid sequence that is at least identical to 96% of the amino acid sequence listed in SEQ ID NO. 2.

2. The factor-according to claim 1 with an amino acid sequence that is at least 96.5%, identical to the amino acid sequence listed in SEQ ID No. 2.

3. Factor RecA, encoded from a nucleic acid, whose nucleotide sequence is at least 85% identical with the nucleotide sequence listed in SEQ ID NO. 1.

4. The factor according to claim 3, encoded from a nucleic acid, whose nucleotide sequence is at least 87.5 identical of the nucleotide sequence listed in SEQ ID No. 1.

5. Nucleic acid encoding for a factor RecA, whose nucleotide sequence is at least 85% identical with the nucleotide sequence listed in SEQ ID NO. 1.

6. The nucleic acid according to claim 5, whose nucleotide sequence is at least 87.5% identical to the nucleotide sequence listed in SEQ ID NO. 1.

7. The nucleic acid according to claim 5, encoding for a factor RecA, wherein the amino acid sequence is at least identical to 96% of the amino acid sequence listed in SEQ ID NO: 2.

8. A method of functionally inactivating the gene recA in a gram-positive bacterium that is not Bacillus megaterium, said method comprising the step of inactivating said recA gene with a nucleic acid sequence that encodes a factor RecA.

9. The method of claim 8, wherein a nucleic acid that encodes for a non-active protein is introduced with a point mutation.

10. The method of claim 8, wherein a nucleic acid with a deletion mutation or insertion mutation is employed, preferably comprising each of the boundary sequences that comprise at least 70 to 150 nucleic acid positions of the region encoding for the protein.

11. The method of claim 8, wherein nucleic acids with a total of two nucleic acid segments are employed that each comprise at least 70 to 150 nucleic acid positions and thereby at least partially, preferably completely flank the region encoding for the protein.

12. (canceled)

13. The method of claim 8, wherein the gram-positive bacterium is naturally capable of sporulation and a gene from the phase IV of the sporulation is simultaneously functionally inactivated with recA.

14. The method of claim 13, wherein the inactivated gene from the phase IV sporulation in the nomenclature of B. subtilis concerns one of the genes spoIVA, spoIVB, spoIVCA, spoIVCB, spoIVFA, spoIVFB or yqfD or homologue thereof.

15. The method of claim 13, wherein exactly one gene from the phase IV of the sporulation is functionally inactivated.

16. The method of claim 14, wherein the functional inactivation of the genes spoIVA, spoIVB, spoIVCA, spoIVCB, spoIVFA, spoIVFB, yqfD or spoIV or of each of their homologous genes occurs with the help of the sequences SEQ ID NO. 3, 5, 7, 9, 11, 13, 15 or 17 or parts thereof.

17. A gram-positive bacterium that is not Bacillus megaterium in which the gene recA is functionally inactivated.

18. The gram-positive bacterium according to claim 17, wherein the functional inactivation is effected through point mutagenesis, partial deletion or insertion or total deletion of the encoding region for the complete protein.

19. The gram-positive bacterium of claim 17, wherein the functional inactivation is effected through a nucleic acid which comprises a nucleotide sequence at least 85% identical to SEQ ID NO: 1.

20. The gram-positive bacterium of claim 17, wherein said bacterium is naturally capable of sporulation and by which a gene from the phase IV of the sporulation is simultaneously functionally inactivated with recA.

21. The gram-positive bacterium according to claim 20, wherein the inactivated gene from the phase IV of the sporulation in the nomenclature of B. subtilis concerns one of the genes spoIVA, spoIVB, spoIVCA, spoIVCB, spoIVFA, spoIVFB or yqfD or homologue thereof.

22. The gram-positive bacterium of claim 20, wherein exactly one gene from the phase IV of the sporulation is functionally inactivated.

23. The gram-positive bacterium of claim 21, wherein the functional inactivation of the genes spoIVA, spoIVB, spoIVCA, spoIVCB, spoIVFA, spoIVFB, yqfD or spoIV or of each of their homologous genes is effected with the help of the sequences SEQ ID NO. 3, 5, 7, 9, 11, 13, 15 or 17 or parts thereof.

24. The gram-positive bacterium of claim 17, wherein said bacterium is from the genera Clostridium or Bacillus.

25. A process for fermenting a gram-positive bacterium comprising the step of fermenting a gram-positive bacterium of claim 17.

26. The process according to claim 25, wherein said gram-positive bacterium produces a low molecular weight compound or a protein.

27. The process according to claim 26, wherein the low molecular weight compound is a natural product, a nutritional supplement or a pharmaceutically relevant compound.

28. The process according to claim 26, wherein the protein is an enzyme.

29. Use of the factor RecA of claim 1, in a molecular biological reaction approach.

30. Use according to claim 29 for stabilizing single stranded DNA in a DNA polymerization, recombination processes in vitro, converting double stranded DNA into single stranded DNA or vice versa.

31. A vector, comprising the nucleic acid of claim 5.

32. The vector according to claim 31, wherein said vector is an expression vector.

33. A process for the manufacture of a factor RecA of claim 1.

34. The process according to claim 33, under addition of the nucleic acid of claim 1 to a host cell.

35. Use of the nucleic acid encoding for a factor RecA of claim 1 for expressing this factor.

36. Use according to claim 34 to manufacture this factor itself, or to modulate molecular biological activities of the cells in recombination processes in vivo.

37. Use of the nucleic acid encoding for the factor for the inactivation of this factor of the gene recA in an in vitro approach through interaction with an associated nucleic acid.

38. (canceled)

39. Use of at least one, preferably at least two nucleic acids orientated against one another according to SEQ ID NO. 25 to 30 for the amplification of an in vivo DNA region enclosed thereby.

40. Use according to claim 39 for the amplification of a recA gene.

41. Use according to claim 39 in the context of a process of claim 8.

42. Use according to claim 39 for the production of a gram-positive bacterium of claim 17.

43. (canceled)

44. (canceled)

45. Use according to claim 39 for the amplification of a spoIV gene.

46. Use according to claim 45 in the context of a process according to claim 13.

47. Use according to claim 45 for the production of a gram-positive bacterium according to claim 20.

48. The method of claim 8, wherein said nucleic acid sequence comprises a nucleotide sequence at least 85% identical to SEQ ID NO: 1.


SEQUENCE LISTING (32 sequences)

SEQ ID NO: 1 - DNA, 1047 bp, Bacillus licheniformis DSM 13
  CDS (1)..(1047)
  gene (1)..(1047): recA
atgagtgatcgtcaggcagccttagatatggcgcttaaacaaatagaaaagcagtttggtaaaggttcgattatgaaactcggcgaacaaactgaaacgagaatttcaacagttccgagcggttctttagcgctcgatgcggctcttggagtgggcggatacccgcgcggccggattattgaagtatacgggcctgaaagctccggtaaaacgacggtggcgcttcatgcgattgccgaagttcagcagcagggcggacaagcggcgttcatcgacgccgacaccgcgcttgatcccgtctatgcacaaaagctgggcgtcaacattgatgagcttttgctgtcacagcctgatacgggcgagcaggcgctcgaaatcgctgaagcccttgtcagaagcggagcggtggatatcgttgtcatcgactctgtagcagcgcttgtgccgaaagctgaaatcgaaggagatatgggggattcccacgtcggtttgcaggccagactgatgtctcaggcgcttcgcaagctttccggagcgatcaataaatcgaagaccatcgcgatctttatcaaccagattcgtgaaaaagtcggtgtcatgtttggaaatcctgagacgacgccaggcggaagagcgctgaaattctactcttctgtccgccttgaagtgcgccgcgcagagcagctgaaacaaggcaacgacgtcatggggaacaagacgaaaatcaaagtcgtgaaaaacaaagtggcacctccattccggacagccgaagtggacattatgtacggggaaggaatttcaaaagaaggggaaatcatcgacctcggaacagagcttgacatcgttcaaaagagcggtgcatggtactcttatcaggaggaacgccttggacaaggccgtgaaaacgccaaacagttcctgaaagaaaacaaggatatccttttgatgattcaagagcagatccgggagcactacggtttggatactggaggcgctgctcctgcacaggaagacgaggcccaagctcaggaagaactcgagttttaa

SEQ ID NO: 2 - protein, 348 aa, Bacillus licheniformis DSM 13
MSDRQAALDMALKQIEKQFGKGSIMKLGEQTETRISTVPSGSLALDAALGVGGYPRGRIIEVYGPESSGKTTVALHAIAEVQQQGGQAAFIDADTALDPVYAQKLGVNIDELLLSQPDTGEQALEIAEALVRSGAVDIVVIDSVAALVPKAEIEGDMGDSHVGLQARLMSQALRKLSGAINKSKTIAIFINQIREKVGVMFGNPETTPGGRALKFYSSVRLEVRRAEQLKQGNDVMGNKTKIKVVKNKVAPPFRTAEVDIMYGEGISKEGEIIDLGTELDIVQKSGAWYSYQEERLGQGRENAKQFLKENKDILLMIQEQIREHYGLDTGGAAPAQEDEAQAQEELEF

SEQ ID NO: 3 - DNA, 1792 bp, Bacillus licheniformis
  CDS (140)..(1336)
  gene (1)..(1792): spoIV
  misc_feature (140)..(142): First codon translated as Met.
ggctgatgctcaaacaggggcagtgcatcattcaaggcaaagactttgtcatcaaaacgattttgcctgaggaaattctgcttgaaggcacgattgagcttgtccgctatatcgattcataagtcggggggaaagaagcgtgaagaataaatggctttcttttttttcaggaaagatccagcttaagataacgggaaaagggatcgaacggttattaaatgaatgcaccaggcgcaacatcccgatgtttaatgtaaagaaaaagaaagacgccgtctttctttatattccgctttctgatgtacatgccttccggaaggtcatcagaggcttcgactgcaagtgcaggttcatcaaacgaaaagggtttcctttcctcgtgcagaagtctaaacggaatagcggcttcacttttggagttgctgcattttttatcatcatgctcctattgtccaacatgctttggaaaattgatattacaggagccaatccggagacagaacatcaaatcaaacagcaattggatcaaatcggcgtcaaaaaaggccgctttcagttttcaatgctgaccccggaaaaaattcagcaggcgctcacaaagcgggtcgaaaacatcacttgggtgggtattgagttaaacggcaccgcccttcacatgaaagtcgttgaaaagaatgaacctgacaaagaaaaatatatcggtccgaggcacatcgtcgccaaaaaaggggcgaccatctcgaaaaagttcgtggaaaaaggcgagccgctcgtcacggtgaaccagcacgttgaaaaagggcaaatgctcgtttccgggctgatcggaagcgaagaggaaaagcaaaaagtcggagcaaaagggaaaatctacggtgaaacctggtacaagtcaacagtaacggttcctcttgagacatcatttgacgtttttacgggtaaagtaaggacaagtcacaagctatccctcggatcattttccgtgccgatctggggcttttcatttaaaaaagaagacttctcgcgcccgaagacggagaccgaaaacccctcgctgcattttatgaattttaagcttcctgtcgcttatgaaaaggagcatatgagggagagcgaacaaatcaaaagggtgtactcgaaaaaagaagcagttcttgaaggaatcgaaatgggaaaaagagacatcaggaaaaaaatcggcagcgacgggaacattatcagtgaaaaagttttgcacgaaacgagcgagaatggcaaagttaaattgatcatcctttaccaggttattgaagacattgttcaaacaacaccaattgttcaggagactaaagaatgacagaacacttacttgcaattcatcagcaactggaaagtccgaatgaggctcaaacgctgtttgggaaccaggattcccatttgaagttgatggaggaagagctgaacatttcaattgtcacgcgcggagaaaccgtgtatgtgacaggagatgaagaaacgtttgaaatcgcggacagcctgcttgcctctctcctaaatctgatccgcaaaggaatcgagatatccgaacgcgatgtcttgtatgcgatcaagatggcgaaaaagcagaagcttgagttttttgaaagcatgtatgaagaggaaattacgaaaaacgccaaaggaaaaccgatcagagtcaaaaccatcggtcaaagagaatacatcgccgccatgaaaaggcacgacttaatcttcggcatcggcccagcaggaacggggaaaacctatttggctgtcgtaaaggccgttcatg

SEQ ID NO: 4 - protein, 398 aa, Bacillus licheniformis
VKNKWLSFFSGKIQLKITGKGIERLLNECTRRNIPMFNVKKKKDAVFLYIPLSDVHAFRKVIRGFDCKCRFIKRKGFPFLVQKSKRNSGFTFGVAAFFIIMLLLSNMLWKIDITGANPETEHQIKQQLDQIGVKKGRFQFSMLTPEKIQQALTKRVENITWVGIELNGTALHMKVVEKNEPDKEKYIGPRHIVAKKGATISKKFVEKGEPLVTVNQHVEKGQMLVSGLIGSEEEKQKVGAKGKIYGETWYKSTVTVPLETSFDVFTGKVRTSHKLSLGSFSVPIWGFSFKKEDFSRPKTETENPSLHFMNFKLPVAYEKEHMRESEQIKRVYSKKEAVLEGIEMGKRDIRKKIGSDGNIISEKVLHETSENGKVKLIILYQVIEDIVQTTPIVQETKE

SEQ ID NO: 5 - DNA, 1594 bp, Bacillus subtilis
  CDS (201)..(1397)
  gene (1)..(1594): yqfD
  misc_feature (201)..(203): First codon translated as Met.
gacttcatatctacatagaaaaccacagaggccttttgcttttcagtgagaatgaagtgcggctgatgctgaagcagggccagtgcatcatatctggtaaaaattttgtcatcaaggcgattcttccggaagagatacttttggagggtacgattgatgtcgttcgatatgttgagtcataaagccgagggggaaatgttgtgaaaaataaatggctgtcttttttttcgggtaaggtccagcttgaattgacgggaagagggattgagcggctccttaatgaatgcacaaaacaggggattccggtctttcatgtcaaaaaaaagaaagaagccgtatcgttatatatacagcttcaggatgtacatgcctttcggcgggtaagaagtaaatttaaatgtaaagcccgatttatcaatcggaagggatttcccttcctgttgctgaaatcaaagctgaatatagggtttacgatcggttttgcgatttttttcattcttttgtttttgctgtccaatatggtgtggaaaattgatgtgacaggcgctaagcctgaaacagaacatcaaatgaggcagcatcttaatgaaatcggcgtcaaaaagggccgtctgcagtttttaatgatgtcgcccgaaaaaatacagaaatcattaaccaatggaatagacaatatcacttgggtcggagttgatctgaaggggacgaccattcatatgaaagttgtggagaaaaatgagcccgaaaaagaaaaatatgttagcccgcgcaatattgtcgccaaaaagaaagcaaccattacgagaatgtctgtgcaaaaaggacagcccatggccgccatacacgatcatgttgaaaagggacagctgcttgtttcgggactgatcggcagcgaagaccatcagcaggaagtcgcctcaaaagcagaaatttatggagaaacctggtatagatcagaagtgacagtcccgcttgaaacattatttaacgtctatacgggcaaagtaaggacaaagcacaagctttcttttggttctttggcaatcccgatctgggggatgacgtttaaaaaagaggaattgaagcatccaaaaacagaacaagaaaagcattcgcttcattttctcggatttaagctccctgtatcctatgtcaaagagcaaacgagagaaagtgaagaggctttgcgaaaatatacaaaagaagaagcagttcaagaaggcattaaattgggtaaacaggatgtagaggataaaataggcgaaaacggcgaggtgaaaagtgaaaaagttttgcaccagactgttgagaatggtaaagtaaagttgattattctctaccaagttatagaagatatcgttcaaaccacacctattgtcagggagactgaagaatgacagaacatttacttgcgatgaatcaaaaactgaaaaacccggacgaggcgctttcactcttcgggaaccaagattcttttttgaaattgatggagaaagatctgaatttaaatatcattacgcgcggcgagacgatttatgtttcaggcgatgatgaatcgtttcagattgcagacaggctgctgggatcgctcctc

SEQ ID NO: 6 - protein, 398 aa, Bacillus subtilis
VKNKWLSFFSGKVQLELTGRGIERLLNECTKQGIPVFHVKKKKEAVSLYIQLQDVHAFRRVRSKFKCKARFINRKGFPFLLLKSKLNIGFTIGFAIFFILLFLLSNMVWKIDVTGAKPETEHQMRQHLNEIGVKKGRLQFLMMSPEKIQKSLTNGIDNITWVGVDLKGTTIHMKVVEKNEPEKEKYVSPRNIVAKKKATITRMSVQKGQPMAAIHDHVEKGQLLVSGLIGSEDHQQEVASKAEIYGETWYRSEVTVPLETLFNVYTGKVRTKHKLSFGSLAIPIWGMTFKKEELKHPKTEQEKHSLHFLGFKLPVSYVKEQTRESEEALRKYTKEEAVQEGIKLGKQDVEDKIGENGEVKSEKVLHQTVENGKVKLIILYQVIEDIVQTTPIVRETEE

SEQ ID NO: 7 - DNA, 1876 bp, Bacillus subtilis
  CDS (201)..(1679)
  misc_feature (201)..(203): First codon translated as Met.
  gene (1)..(1876): spoIVA
atgatatgaaaaaggaatgaacctttctcccttgcatacaaatagggagaaaggtttttttatattaatagattgaggatgagaaattttctaaagatgtcatattcaaataggacaacgtcatacacatatagtgtcctgtgtttgattgaaagagcttaataaaattgaaaaggataggaagtccgggaggggatcacttggaaaaggtcgatattttcaaggatatcgctgaacgaacaggaggcgatatatacttaggagtcgtaggtgctgtccgtacaggaaaatccacgttcattaaaaaatttatggagcttgtggtgctcccgaatatcagtaacgaagcagaccgggcccgagcgcaggatgaactgccgcagagcgcagccggcaaaaccattatgactacagagcctaaatttgttccgaatcaggcgatgtctgttcatgtgtcagacggactcgatgtgaatataagattagtagattgtgtaggttacacagtgcccggcgctaaaggatatgaagatgaaaacgggccgcggatgatcaatacgccttggtacgaagaaccgatcccatttcatgaggctgctgaaatcggcacacgaaaagtcattcaagaacactcgaccatcggagttgtcattacgacagacggcaccattggagatatcgccagaagtgactatatagaggctgaagaaagagtcattgaagagctgaaagaggttggcaaaccttttattatggtcatcaactcagtcaggccgtatcacccggaaacggaagccatgcgccaggatttaagcgaaaaatatgatatcccggtattggcaatgagtgtagagagcatgcgggaatcagatgtgctgagtgtgctcagagaggccctctacgagtttccggtgctagaagtgaatgtcaatctcccaagctgggtaatggtgctgaaagaaaaccattggttgcgtgaaagctatcaggagtccgtgaaggaaacggttaaggatattaaacggctccgggacgtagacagggttgtcggccaattcagcgagtttgaattcattgaaagtgccggattagccggaattgagctgggccaaggggtggcagaaattgatttgtacgcgcctgatcatctatatgatcaaatcctaaaagaagttgtgggcgtcgaaatcagaggaagagaccatctgcttgagctcatgcaagacttcgcccatgcgaaaacagaatatgatcaagtgtctgatgccttaaaaatggtcaaacagacgggatacggcattgcagcgcctgctttagctgatatgagtctcgatgagccggaaattataaggcagggctcgcgattcggtgtgaggctgaaagctgtcgctccgtcgatccatatgatcaaagtagatgtcgaaagcgaattcgccccgattatcggaacggaaaaacaaagtgaagagcttgtacgctatttaatgcaggactttgaggatgatccgctctccatctggaattccgatatcttcggaaggtcgctgagctcaattgtgagagaagggattcaggcaaagctgtcattgatgcctgaaaacgcacggtataaattaaaagaaacattagaaagaatcataaacgaaggctctggcggcttaatcgccatcatcctgtaataccggtagacctctttatagaatgggaggtcttttttctttgctcttaataatggaaaaggatcaaggaataggatgaaaaaaggaaaaaaaggaatattcgttcggtaaatcaccttaaatccttgacgagcaagggattgacgctttaaaatgcttgatatggctttttatatgtgttactctacatacagaaa

SEQ ID NO: 8 - protein, 492 aa, Bacillus subtilis
LEKVDIFKDIAERTGGDIYLGVVGAVRTGKSTFIKKFMELVVLPNISNEADRARAQDELPQSAAGKTIMTTEPKFVPNQAMSVHVSDGLDVNIRLVDCVGYTVPGAKGYEDENGPRMINTPWYEEPIPFHEAAEIGTRKVIQEHSTIGVVITTDGTIGDIARSDYIEAEERVIEELKEVGKPFIMVINSVRPYHPETEAMRQDLSEKYDIPVLAMSVESMRESDVLSVLREALYEFPVLEVNVNLPSWVMVLKENHWLRESYQESVKETVKDIKRLRDVDRVVGQFSEFEFIESAGLAGIELGQGVAEIDLYAPDHLYDQILKEVVGVEIRGRDHLLELMQDFAHAKTEYDQVSDALKMVKQTGYGIAAPALADMSLDEPEIIRQGSRFGVRLKAVAPSIHMIKVDVESEFAPIIGTEKQSEELVRYLMQDFEDDPLSIWNSDIFGRSLSSIVREGIQAKLSLMPENARYKLKETLERIINEGSGGLIAIIL

SEQ ID NO: 9 - DNA, 1675 bp, Bacillus subtilis
  CDS (201)..(1478)
  gene (1)..(1675): spoIVB
cggatcaagtcaaaacaactgggtaagctgcgcgagaagcgcagcttatttttttcgtgcacatccattcgttcatcagtatatccaatgtttttcttcatatgacagttataaataagccgtcagaaggcaaaattaaatgatgtagcagcaagtcataaagaaggtgtgggataggagcgaggagagtgaagtagtgaatgcccgataacatcagaaaagcagtaggtttaattctccttgtttcgttattaagtgtaggtttatgcaaaccgctaaaagaatatttactgattccaacgcaaatgagagtatttgaaacccaaacacaagcgattgaaacgagtttatcggtaaatgctcagacatcagaatcctcagaagcgtttacagtaaagaaagatccgcatgaaatcaaggtgacgggcaaaaaatcaggtgagtcagaattggtatatgatcttgccggatttccaattaaaaaaacaaaagtgcatgttcttcctgatttaaaagttatacctggcggacaatcaatcggtgtaaaacttcattccgtcggtgttcttgtcggatttcatcaaatcaatacaagtgaaggcaaaaaatctccgggagaaacggcaggaattgaagcgggcgacatcattattgagatgaatggacagaaaattgaaaaaatgaatgatgtagccccatttattcaaaaggctgggaaaactggtgaatctttagacttactgatcaaacgtgataaacagaaaatcaaaacgaagctgatcccagaaaaggatgaaggagaaggcaaatacagaatcgggttatatatcagagattctgctgctggcatcggcactatgaccttttatgaaccgaaaacaaaaaaatacggagcacttggccacgtgatttccgatatggacacaaagaaaccaattgtagtggagaatggagaaatcgttaaatccactgtaacatcaattgaaaaagggacaggcggtaatccgggagaaaaactggcgcgattttcctcagaacgcaaaacgatcggggatattaacagaaacagcccgtttgggattttcggcacactgcatcagccgattcaaaacaacatatcagatcaagcattgccggttgcgttttctaccgaagtcaaaaaagggccggctgaaattttaacggttattgatgatgacaaagtagaaaaattcgatattgaaatcgtcagcacaacgccgcaaaaattccctgcgacaaaaggaatggtgttgaaaattaccgatccaagactgttgaaagaaacaggaggcatcgtacaggggatgagcggaagcccgatcattcaaaatggaaaagtgatcggtgctgtcacccatgtatttgtaaatgacccgacaagcggctacggtgttcatattgaatggatgctgtcagaagcaggaatcgatatttatggaaaagaaaaagcaagctgactgccggagtttccggcagtttttttattttgatccctcttcacttctcagaatacatacggtaaaatatacaaaagaagatttttcgacaaattcacgtttccttgtttgtcaaatttcatttttagtcgaaaaacagagaaaaacatagaataacaaagatatgccactaatattggtgattatgatttttttag

SEQ ID NO: 10 - protein, 425 aa, Bacillus subtilis
MPDNIRKAVGLILLVSLLSVGLCKPLKEYLLIPTQMRVFETQTQAIETSLSVNAQTSESSEAFTVKKDPHEIKVTGKKSGESELVYDLAGFPIKKTKVHVLPDLKVIPGGQSIGVKLHSVGVLVGFHQINTSEGKKSPGETAGIEAGDIIIEMNGQKIEKMNDVAPFIQKAGKTGESLDLLIKRDKQKIKTKLIPEKDEGEGKYRIGLYIRDSAAGIGTMTFYEPKTKKYGALGHVISDMDTKKPIVVENGEIVKSTVTSIEKGTGGNPGEKLARFSSERKTIGDINRNSPFGIFGTLHQPIQNNISDQALPVAFSTEVKKGPAEILTVIDDDKVEKFDIEIVSTTPQKFPATKGMVLKITDPRLLKETGGIVQGMSGSPIIQNGKVIGAVTHVFVNDPTSGYGVHIEWMLSEAGIDIYGKEKAS

SEQ ID NO: 11 - DNA, 1900 bp, Bacillus subtilis
  CDS (201)..(1703)
  gene (1)..(1900): spoIVCA
  misc_feature (201)..(203): First codon translated as Met.
ttttgcatattcattgaaacgtttaataacactatagtttaatttaaaatctcctcatttggacaaacagctgttacatagcattacccaaggggtgatgcattttatgaaagtgataatcatcgagggaccgcaagctgacaaatgcattaacgattgctatcattatttaataaaactttataggaaggagattcagggtgatagcaatatatgtaagggtatcgaccgaggaacaagcgatcaagggatcgagcatcgacagccaaatcgaggcctgtataaagaaagcagggactaaagatgtgctgaagtatgcagatgaaggattttcaggagagcttttagaacgtccggctttgaatcgcttgagggaggatgcaagcaagggacttataagtcaagtcatttgttacgatcctgaccgtctttctcggaaattaatgaatcagctaatcattgatgacgaattgcgaaagcgaaacatacctttgatttttgtaaatggtgaatacgccaattctccagaaggtcaattgtttttcgcaatgcgcggggcaatctcagaatttgaaaaagccaaaatcaaagaacggacatcaagcggccgacttcaaaaaatgaaaaaaggcatgatcattaaagattctaaactatatggctataaatttgttaaagagaaaagaactcttgagatattagaagaggaagcaaaaatcattcggatgatttttaactatttcaccgatcataaaagcccttttttcggcagagtaaatggtattgctctacatttaactcagatgggggttaaaacaaaaaaaggcgccaaagtatggcacaggcaggttgttcggcaaatattaatgaactcttcctataagggtgaacatagacagtataaatatgatacagagggttcctatgtttcaaagcaggcagggaacaaatctataattaaaataaggcctgaagaagaacaaatcactgtgacaattccagcaattgttccagctgaacaatgggattatgctcaagaactcttaggtcaaagtaaaagaaaacacttgagtatcagccctcacaattacttgttatcgggtttggttagatgcggaaaatgcggaaataccatgacagggaagaaaagaaaatcacatggtaaagactactatgtatatacttgccggaaaaattattctggcgcaaaggaccgcggctgcggaaaagaaatgtctgagaataaattgaaccggcatgtatggggtgaaatttttaaattcatcacaaatcctcaaaagtatgtttcttttaaagaggctgaacaatcaaatcacctgtctgatgaattagaacttattgaaaaagagatagagaaaacaaaaaaaggccgcaagcgtcttttaacgctaatcagcctaagcgatgacgatgatttagacatagatgaaatcaaagcacaaattattgaactgcaaaaaaagcaaaatcagcttactgaaaagtgtaacagaatccagtcaaaaatgaaagtcctagatgatacgagctcaagtgaaaatgctctaaaaagagccatcgactattttcaatcaatcggtgcagataacttaactcttgaagataaaaaaacaattgttaactttatcgtgaaagaagttaccattgtggattctgacaccatatatattgaaacgtattaaagaggggtgtatgcaccccccttttgtaattacaatctcattttcaatacacctcgctgcatacgtcgccacctttgtcccttttccagcggaatagctttcaattcctttaataagcccgatcgttccgatggagattaagtcctctgcatcctcacctgtattttcgaactttttcacaatatgggcgaccaagc

SEQ ID NO: 12 - protein, 500 aa, Bacillus subtilis
VIAIYVRVSTEEQAIKGSSIDSQIEACIKKAGTKDVLKYADEGFSGELLERPALNRLREDASKGLISQVICYDPDRLSRKLMNQLIIDDELRKRNIPLIFVNGEYANSPEGQLFFAMRGAISEFEKAKIKERTSSGRLQKMKKGMIIKDSKLYGYKFVKEKRTLEILEEEAKIIRMIFNYFTDHKSPFFGRVNGIALHLTQMGVKTKKGAKVWHRQVVRQILMNSSYKGEHRQYKYDTEGSYVSKQAGNKSIIKIRPEEEQITVTIPAIVPAEQWDYAQELLGQSKRKHLSISPHNYLLSGLVRCGKCGNTMTGKKRKSHGKDYYVYTCRKNYSGAKDRGCGKEMSENKLNRHVWGEIFKFITNPQKYVSFKEAEQSNHLSDELELIEKEIEKTKKGRKRLLTLISLSDDDDLDIDEIKAQIIELQKKQNQLTEKCNRIQSKMKVLDDTSSSENALKRAIDYFQSIGADNLTLEDKKTIVNFIVKEVTIVDSDTIYIETY

SEQ ID NO: 13 - DNA, 868 bp, Bacillus subtilis
  CDS (201)..(671)
  gene (1)..(868): spoIVCB
ttcatccccatccccccatacctttgttcatttcaatgtatgggcgcttgatgaagaatatttttaacatttgaagttagtatgctgcttaccaaagccggactcccccgcgagaaatttcccggtacagacacagacagcctcccggtcacatacatttacatataggcttttgcctacatacttttgtggaggtgacgatggtgacaggtgttttcgcagcgctcggctttgttgttaaagagcttgtctttttagtatcttacgtgaaaaacaatgcctttccacaaccgctctcaagcagcgaagaaaaaaaatacttagagctcatggctaaaggggatgaacatgccagaaacatgctgattgagcataatcttcgcttggtcgcccatattgtgaaaaagttcgaaaatacaggtgaggatgcagaggacttaatctccatcggaacgatcgggcttattaaaggaattgaaagctattccgctggaaaagggacaaaggtggcgacgtatgcagcgaggtgtattgaaaatgagattgtaattacaaaaggggggtgcatacacccctctttaatacgtttcaatatatatggtgtcagaatccacaatggtaacttctttcacgataaagttaacaattgtttttttatcttcaagagttaagttatctgcaccgattgattgaaaatagtcgatggctctttttagagcattttcacttgagctcgtatcatctaggactttcatttttgactggattctgttacacttttcagtaagctgattttgctttttttgcagttcaataatttgtgctttgatttcatctatgtctaaatcatcgtcatcgcttaggctga

SEQ ID NO: 14 - protein, 156 aa, Bacillus subtilis
MVTGVFAALGFVVKELVFLVSYVKNNAFPQPLSSSEEKKYLELMAKGDEHARNMLIEHNLRLVAHIVKKFENTGEDAEDLISIGTIGLIKGIESYSAGKGTKVATYAARCIENEIVITKGGCIHPSLIRFNIYGVRIHNGNFFHDKVNNCFFIFKS

SEQ ID NO: 15 - DNA, 1192 bp, Bacillus subtilis
  CDS (201)..(995)
  gene (1)..(1192): spoIVFA
acaaaggaatgatggctaagattaagtcatttttcggagtaagatcttaatgtgatagaatcaaagagaagaatctgacaaagcatatgctgtgtcaggtttttttttgtttttgcctgctttgttcttgactaaaccgaatatttgccatggacaagacatatgatgtacaaacccaacgaatgcaaaggatgatggcaatgagtcacagagcagatgaaatcagaaaacgattagagaaaagaagaaagcagctttccggctcaaaacgtttctctactcagacagtttctgaaaagcagaaacccccgtcctgggtgatggtaacggatcaggaaaagcatggaacacttccggtctacgaagataacatgccaacattcaacggaaaacacccattggtgaaaacagattcaattatcctgaaatgtcttctgtcggcctgccttgttctcgtttcagctatagcctataaaacaaacattggacccgtcagtcagattaaacccgccgtagccaaaacctttgaaactgaatttcaatttgcttcagcaagccattggttcgaaaccaaattcggaaatccgcttgctttcctggctcctgaacacaaaaataaggaacagcagattgaagtaggcaaagatctgatcgcgcctgcatccgggaaagtacagcaggattttcaggacaatggggaaggaattaaagtcgaaacaagcagtgataagattgatagcgtaaaagaaggctatgtggttgaagtcagcaaagacagccaaacgggactgacggttaaggtgcagcatgctgacaacacctatagtatctatggcgagctcaaagatgtggatgttgctttatatgattttgtggataaaggcaaaaagctcggttcgattaagcttgatgatcataataaaggggtctattattttgccatgaaagacggcgataaatttattgatccgattcaggtgatttcatttgaataaatggctcgaccttatcttaaagatccatgtgcatccttttctttggattattgcggcgctgggcttgctcacaggccatatgaaagcattattatgtctgctcctgattgtattgattcatgagctggggcatgctgctctggctgtgtttttttcttggagaatcaagcgtgtttttttgctgccgtttggcggaa

SEQ ID NO: 16 - protein, 264 aa, Bacillus subtilis
MSHRADEIRKRLEKRRKQLSGSKRFSTQTVSEKQKPPSWVMVTDQEKHGTLPVYEDNMPTFNGKHPLVKTDSIILKCLLSACLVLVSAIAYKTNIGPVSQIKPAVAKTFETEFQFASASHWFETKFGNPLAFLAPEHKNKEQQIEVGKDLIAPASGKVQQDFQDNGEGIKVETSSDKIDSVKEGYVVEVSKDSQTGLTVKVQHADNTYSIYGELKDVDVALYDFVDKGKKLGSIKLDDHNKGVYYFAMKDGDKFIDPIQVISFE

SEQ ID NO: 17 - DNA, 1264 bp, Bacillus subtilis
  CDS (201)..(1067)
  gene (1)..(1264): spoIVFB
  misc_feature (201)..(203): First codon translated as Met.
actgacggttaaggtgcagcatgctgacaacacctatagtatctatggcgagctcaaagatgtggatgttgctttatatgattttgtggataaaggcaaaaagctcggttcgattaagcttgatgatcataataaaggggtctattattttgccatgaaagacggcgataaatttattgatccgattcaggtgatttcatttgaataaatggctcgaccttatcttaaagatccatgtgcatccttttctttggattattgcggcgctgggcttgctcacaggccatatgaaagcattattatgtctgctcctgattgtattgattcatgagctggggcatgctgctctggctgtgtttttttcttggagaatcaagcgtgtttttttgctgccgtttggcggaacggtcgaagtggaagagcacgggaatcggccgttaaaggaagagtttgcggtcattattgccggacctcttcagcacatctggcttcagtttgccgcctggatgcttgcagaagtctcagtgattcatcagcatacctttgaactcttcaccttttataatctttctatcttatttgtcaatttactgccgatctggccgctggatggaggaaaactgttatttttgttgttttccaaacagctgccttttcaaaaggctcaccggcttaatctaaaaacgtcgctctgcttctgcctgctgctcgggtgctgggttttattcgtgattcctctgcaaatcagcgcatgggttttgtttgtctttctggctgtttccttgtttgaggaatatcggcaaaggcactatatccatgtgagatttctcctcgaaaggtattacggaaaaaacagggagcttgagaaacttctgccgctgacagtaaaggcggaggataaagtctatcatgtgatggccgagttcaaacgtggctgtaagcatccgattattatagaaaaatcaggccaaaagctcagccagcttgacgagaatgaagtgctgcacgcttactttgccgataagcggacgaattcttccatggaggaactgcttctgccctactaaaactgattgacaaacgccttgtattttggtatattttttaatgttatggatgtagcaccattgctacaaccgctcagtacaggtgttaagagcttttacagccccctggtatctggcgagtcttagtctaataggaggtgcagagaatgtacgcaatcattaaaacaggcggtaaacaaatcaaagttgaagaaggc

SEQ ID NO: 18 - protein, 288 aa, Bacillus subtilis
LNKWLDLILKIHVHPFLWIIAALGLLTGHMKALLCLLLIVLIHELGHAALAVFFSWRIKRVFLLPFGGTVEVEEHGNRPLKEEFAVIIAGPLQHIWLQFAAWMLAEVSVIHQHTFELFTFYNLSILFVNLLPIWPLDGGKLLFLLFSKQLPFQKAHRLNLKTSLCFCLLLGCWVLFVIPLQISAWVLFVFLAVSLFEEYRQRHYIHVRFLLERYYGKNRELEKLLPLTVKAEDKVYHVMAEFKRGCKHPIIIEKSGQKLSQLDENEVLHAYFADKRTNSSMEELLLPY

SEQ ID NO: 19 - DNA, 29 bp, Artificial sequence
  Synthetic PCR-Primer spo1
ggctgatgctcaaacaggggcagtgcatc

SEQ ID NO: 20 - DNA, 24 bp, Artificial Sequence
  Synthetic PCR-Primer spo2
catgaacggcctttacgacagcca

SEQ ID NO: 21 - DNA, 25 bp, Artificial Sequence
  Synthetic PCR-Primer spo3
gtcatcaaaacgattttgcctgagg

SEQ ID NO: 22 - DNA, 26 bp, Artificial Sequence
  Synthetic PCR-Primer spo4
atgttctgtcccgggattggctcctg

SEQ ID NO: 23 - DNA, 29 bp, Artificial Sequence
  Synthetic PCR-Primer spo6
gttttgactctgatcggaattctttggcg

SEQ ID NO: 24 - DNA, 22 bp, Artificial Sequence
  Synthetic PCR-Primer spo7
gcacgaaacgagcgagaatggc

SEQ ID NO: 25 - DNA, 26 bp, Artificial Sequence
  Synthetic PCR-Primer recA1
ggaattcggcatcagcttcactggag

SEQ ID NO: 26 - DNA, 29 bp, Artificial Sequence
  Synthetic PCR-Primer recA2
gctatgtcgactataccttgtttatgcgg

SEQ ID NO: 27 - DNA, 21 bp, Artificial Sequence
  Synthetic PCR-Primer recA3
gacctcggaacagagcttgac

SEQ ID NO: 28 - DNA, 30 bp, Artificial Sequence
  Synthetic PCR-Primer recA4
tcaaactgcagtcattaagagaatggatgg

SEQ ID NO: 29 - DNA, 23 bp, Artificial Sequence
  Synthetic PCR-Primer recA5
aagcttacggtttaacgtttctg

SEQ ID NO: 30 - DNA, 26 bp, Artificial Sequence
  Synthetic PCR-Primer recA6
acacaaacgaattgaaagtgtcagcg

SEQ ID NO: 31 - DNA, 1557 bp, Bacillus licheniformis A
  CDS (369)..(1415)
  gene (1)..(1557): recA
gatatcggcatcagcttcactggagtagccgggccgaatacgcaagaaggccatccggctggaaaggtgtttatcggcatctccgtgaaggaccaggctgaggaagcgttcgaatttcagttcgccggatccaggtctgcggtgcggaagcgttctgccaaatacggctgccatctgttgctgaaaatgatggaaaaataagcggaaaccggattttcggaatatctttctttcgaaaaaggccattccattttaggagatcgatttttcctcttaaaaaaatcgaatatgcgttcgcttttttcttggcaaatccgcataaacaaggtatagtagatatagcggaagtgataaaggaggaaaatagaatgagtgatcgtcaggcagccttagatatggcgcttaaacaaatagaaaagcagtttggtaaaggttcgattatgaaactcggcgaacaaactgaaacgagaatttcaacagttccgagcggttctttagcgctcgatgcggctcttggagtgggcggatacccgcgcggccggattattgaagtatacgggcctgaaagctccggtaaaacgacggtggcgcttcatgcgattgccgaagttcagcagcagggcggacaagcggcgttcatcgacgccgaacacgcgcttgatcccgtctatgcacaaaagctgggcgtcaacattgatgagcttttgctgtcacagcctgatacgggcgagcaggcgctcgaaatcgctgaagcccttgtcagaagcggagcggtggatatcgttgtcatcgactctgtagcagcgcttgtgccgaaagctgaaatcgaaggagatatgggggattcccacgtcggtttgcaggccagactgatgtctcaggcgcttcgcaagctttccggagcgatcaataaatcgaagaccatcgcgatctttatcaaccagattcgtgaaaaagtcggtgtcatgtttggaaatcctgagacgacgccaggcggaagagcgctgaaattctactcttctgtccgccttgaagtgcgccgcgcagagcagctgaaacaaggcaacgacgtcatggggaacaagacgaaaatcaaagtcgtgaaaaacaaagtggcacctccattccggacagccgaagtggacattatgtacggggaaggaatttcaaaagaaggggaaatcatcgacctcggaacagagcttgacatcgttcaaaagagcggtgcatggtactcttatcaggaggaacgccttggacaaggccgtgaaaacgccaaacagttcctgaaagaaaacaaggatatccttttgatgattcaagagcagatccgggagcactacggtttggatactggaggcgctgctcctgcacaggaagacgaggcccaagctcaggaagaactcgagttttaatcatgaaacgtgtgaaaggctgccggcccgatcggcagccttttactttattcttcgctttcaggcgcttctcttccatccattctcttaatgagggcagtttgaaaggcgtttaatccagaaacgttaagaccgtaagctt

SEQ ID NO: 32 - protein, 348 aa, Bacillus licheniformis A
MSDRQAALDMALKQIEKQFGKGSIMKLGEQTETRISTVPSGSLALDAALGVGGYPRGRIIEVYGPESSGKTTVALHAIAEVQQQGGQAAFIDAEHALDPVYAQKLGVNIDELLLSQPDTGEQALEIAEALVRSGAVDIVVIDSVAALVPKAEIEGDMGDSHVGLQARLMSQALRKLSGAINKSKTIAIFINQIREKVGVMFGNPETTPGGRALKFYSSVRLEVRRAEQLKQGNDVMGNKTKIKVVKNKVAPPFRTAEVDIMYGEGISKEGEIIDLGTELDIVQKSGAWYSYQEERLGQGRENAKQFLKENKDILLMIQEQIREHYGLDTGGAAPAQEDEAQAQEELEF